(19) United States
(12) Patent Application Publication
(10) Pub. No.: US 2022/0331219 A1
(43) Pub. Date: Oct. 20, 2022

(54) METHODS AND COMPOSITIONS FOR PROVIDING SKIN CARE BENEFITS

(71) Applicant: DANISCO US INC., Palo Alto, CA (US)

(72) Inventors: Heli ANGLENIUS, KIRKKONUMMI (FI); Carole GHERARDI, SAVIGNY (FR); Laura Tiina Maria HUUSKONEN, Espoo (FI); Juho JÄRVINEN, KANTVIK (FI); Henrik Max JENSEN, BRABRAND (DK); Hannu KOIVIKKO, KANTVIK (FI); Jyrki KUUSISTO, VANTAA (FI); Tero Tuomas MENTUNEN, KANTVIK (FI); Juha NURMI, KANTVIK (FI); Piera Mattia PERICU, MANNHEIM (DE); Pertti Matias SÄRELÄ, KANTVIK (FI); Jani SIITONEN, KANTVIK (FI); Kirsti TIIHONEN, HELSINKI (FI); Gregory M. WHITED, PALO ALTO, CA (US)

(21) Appl. No.: 17/634,620

(22) PCT Filed: Aug. 25, 2020
(86) PCT No.: PCT/US2020/047720

§ 371 Date: Feb. 11, 2022

Related U.S. Application Data
(60) Provisional application No. 62/892,760, filed on Aug. 28, 2019.

(51) Int. Cl. A61K 8/49; A61K 8/365; A61Q 17/00; A61Q 19/00; A61Q 19/10; A61Q 19/08

(52) U.S. Cl. CPC A61K 8/498; A61K 8/365; A61Q 17/00; A61Q 19/007; A61Q 19/10; A61Q 19/08; A61K 2800/28

(57) ABSTRACT

Compositions comprising mevalonolactone, mevalonic add, mevalonate, salts of mevalonic acid, mevalonolactone monohydrate, or any combination thereof and methods of use are provided for topical application for skin care, skin supplement, hair care, and oral care. More specifically, the present disclosure is directed towards compositions comprising mevalonolactone, mevalonic acid, mevalonate, salts of mevalonic acid, mevalonolactone monohydrate, or any combination thereof for providing a skin care benefit and/or multiple skin care benefits.

This application claims the benefit of U.S. Provisional Application No. 62/892,760, filed Aug. 28, 2019, U.S. Provisional Application No. 63/012,492, filed Apr. 20, 2020 each of which incorporated by reference herein in their entireties.

### FIELD

The present disclosure relates to compositions comprising mevalonolactone, mevalonic acid, mevalonate, salts of mevalonic acid, mevalonolactone monohydrate, or any combination thereof, and methods of use for skin care, skin supplement, hair care and oral care. More specifically, the present disclosure is directed towards skin care compositions comprising mevalonolactone, mevalonic acid, mevalonate, salts of mevalonic acid, mevalonolactone monohydrate, or any combination thereof, for providing a skin care benefit and/or multiple skin care benefits.

### BACKGROUND

The known processes for isolation and purification of organic acid from solutions such as fermentation broth is very complex and commonly involves an organic solvent extraction step or solid phase absorption.

An important aspect for the industrial use of organic acids such as carboxylic acids which are generated by fermentation of carbohydrate-containing substrates using various microorganisms is the cost-effectiveness and efficiency of removing and purifying the organic acid or salt or lactones thereof from these aqueous fermentation solutions, which contain not only the organic acid or the organic acid salts or lactones but also further organic acids, other fermentation byproducts, microorganisms and the constituents thereof and also remnants of the substrates, such as sugars, proteins, lipids and other trace organic and inorganic compounds.

A classical way to purify microbially produced organic acids from fermentation broth is to protonate the target acid, by lowering the pH of the broth, and performing a liquid/liquid extraction using a partially miscible organic solvent. (Organic Laboratory Techniques, 3rd Edition pg. 49-67. Ralph Fessenden, Joan Fessenden, Patty Feist 2001, Brooks/Cole).

US2014/0371486 describes a method for purifying carboxylic acids from fermentation broths using solid phase adsorption. The method includes removing biomass and any solids present from the fermentation broth, finely cleaning up the biomass-free and solids-free fermentation broth by nanofiltration, and removing the carboxylic acid from the finely cleaned, biomass-free, and solids free fermentation broth by adsorption to one or more solid phases having tertiary amino groups. KR20180070117 describes a method for producing mevalonolactone from biosynthesized mevalonic acid using phosphoric acid.

U.S. Pat. No. 5,034,105 describes a method of crystallizing succinic acid from an undersaturated solution of a succinic acid salt, subjecting said salt solution to electrodialysis to form a supersaturated solution, followed by crystallizing the supersaturated solution of succinic acid by adding to said solution an effective amount of acetic acid to enhance the crystallization of the succinic acid.

A disadvantage of these methods is that additional substances are supplied to the process, which substances must no longer be present in the target product or the traces of which substances in the target product may lead to limitations in the quality and the applicability of the product. The practical implementation of the methods is also associated in some cases with considerable technical complexity and considerable energy consumption.

Therefore, there remains a need for developing more effective. reliable, environmentally friendly and/or economically feasible processes for purifying and recovering organic acids and salts and lactones thereof, and for producing crystalline forms of organic acids and salts or lactones thereof.

The skin functions as a barrier protecting the organism from drying out as well as protecting the organism against the penetration of external, often harmful, substances.

The human skin consists of two main layers of cells, epidermis and dermis. The epidermis constitutes the outermost layer of the skin and is mainly formed of terminally differentiated keratinocytes and lipids, living dividing keratinocytes located beneath the terminally differentiated ones. The outer layer of the epidermis (Stratum corneum or Horny layer) is the part which is in contact with the environment and the particular structure of the horny layer protects the skin as well as stabilizes its own flexibility by binding a defined amount of water (P. M. Elias, Structure and F nction of the Stratum Corneum Permeability Barrier, Drug Dev. Res. 13, 1988, 97-105). The main function of the epidermis is to form permeability barrier against environmental challenges, such as UV radiation, heat, chemicals, pollution, and pathogens, such as bacteria, fungi, parasites, and viruses. It also protects the body from uncontrolled water evaporation from inside out, maintaining the hydration balance and skin metabolism.

In the dermis, the most abundant cell type, dermal fibroblasts, are responsible of generating the connective tissue by producing extracellular matrix (ECM). This extracellular matrix (ECM) is composed of two main classes of macromolecules: proteoglycans (PGs) and fibrous proteins; the most abundant fibrous proteins are type I collagen fibrils, elastins, laminins and fibronectins (Frantz C, et al., The extracellular matrix at a glance, J. Cell Sci. 2010; 123 (24):4195-4200). During aging the collagen fibrils become fragmented, fibroblasts produce less ECM proteins and more ECM degrading matrix metalloproteinases (MMPs), that leads to imbalance in the ECM (Cole M A, et al., Extracellular matrix regulation of fibroblast function: redefining our perspective on skin aging, J Cell Commun Signal. 2018; 12(1):35-43).

There remains a need to find methods and compositions for providing skin benefits, such as but not limiting to methods and compositions for providing a skin care benefit selected from the group consisting of skin moisturizing, skin exfoliation (also referred to as skin peeling. desquamation, skin shedding, skin resurfacing, skin regeneration, skin renewal, improving epidermal cell turnover, preventing or retarding the appearance of the signs of aging of the skin (anti-aging), reducing the appearance of skin winkles. skin rejuvenation, strengthening the skin barrier function, or any one combination thereof) to a skin.

### SUMMARY

The present disclosure is directed to compositions comprising mevalonolactone, mevalonic acid, mevalonate, salts of mevalonic acid, mevalonolactone monohydrate, or any combination thereof, and methods for topical application for skin care, skin supplement. hair care, and oral care. More specifically, the present disclosure is directed towards compositions comprising mevalonolactone, mevalonic acid, mevalonate, salts of mevalonic acid, mevalonolactone monohydrate, or any combination thereof, for providing a skin care benefit and/or multiple skin care benefits in a subject.

In one embodiment, the composition is a skin care composition for providing at least one skin care benefit in a subject, comprising an effective amount of an active ingredient selected from the group consisting of mevalonoladone, mevalonic acid, mevalonate, salts of mevalonic acid, mevalonolactone monohydrate, and any one combination thereof, wherein said composition provides at least one skin care benefit to a skin of said subject.

In one embodiment, the composition is a skin care composition for providing at least one skin care benefit in a subject, comprising one or more dermatologically or cosmetically acceptable components and an effective amount of an active ingredient selected from the group consisting of mevalonolactone, mevalonic acid, mevalonate, salts of mevalonic acid, mevalonolactone monohydrate, and any one combination thereof, wherein said composition provides at least one skin care benefit to a skin of said subject.

In one embodiment, the method is a method for providing at least one skin care benefit in a subject, comprising contacting a skin of said subject with a skin care composition comprising an active ingredient selected from the group consisting of mevalonolactone, mevalonic acid, mevalonolactone monohydrate, and any one combination thereof.

In one aspect, the at least one skin care benefit is selected from the group consisting of skin moisturizing, skin exfoliation (also referred to as skin peeling, desquamation, skin shedding), skin resurfacing, skin regeneration, skin renewal, improving epidermal cell turnover, preventing or retarding the appearance of the signs of aging of the skin (anti-aging), reducing the appearance of skin wrinkles, skin rejuvenation, strengthening the skin barrier function, and any one combination thereof.

Compositions and methods are also provided for producing crystalline forms of organic acids or salts or lactones thereof from an aqueous solution. In one aspect, crystalline mevalonolactone*monohydrate (MVL*H₂O) and methods to crystalize mevalonoladone*monohydrate (MVL*H₂O) are disclosed.

More specifically, in one aspect the specification provides a method for producing a crystalline form of a salt of mevalonic acid (also referred to as X-MVA) from an aqueous solution, comprising subjecting the aqueous solution comprising said X-MVA to a nanofiltration to produce a permeate and crystallizing said X-MVA from said permeate by water solvent crystallization.

In another aspect, the specification provides a method for producing a water solubilized mevalonolactone from an aqueous solution wherein the method comprises: a) producing a crystalline form of a salt of mevalonic acid from an aqueous solution by subjecting the aqueous solution comprising said salt of mevalonic acid (X-MVA) to a nanofiltration to produce a permeate and crystallizing said salt of mevalonic acid from said permeate by water solvent crystallization to produce crystals of said salt of mevalonic acid; and, b) dissolving the crystals of (a) in water to produce a water solubilized salt of mevalonic acid and subjecting said liquid to cation exchange thereby converting said water solubilized salt of mevalonic acid to water solubilized mevalonolactone.

In yet another aspect, the specification provides a method for producing mevalonolactone from an aqueous solution comprising a salt of mevalonic acid salt (X-MVA), comprising subjecting the aqueous solution comprising said salt of mevalonate to cation exchange thereby converting said aqueous solution comprising a salt of mevalonate to an aqueous solution comprising mevalonolactone (MVL) and optionally producing a MVL of high purity (>90% purity) from said aqueous solution by concentrating said solution, producing mevalonolactonemonohydrate (MVL*H₂O) from said concentrated solution, and dissolving the mevalonolactonemonohydrate (MVL*H₂O) crystals in water to obtain the highly pure MVL solution.

Additional embodiments of the methods and compositions of the present disclosure are shown herein.

### DETAILED DESCRIPTION

Compositions and methods are provided for topical applications for skin care, skin supplement, hair care, oral care, comprising mevalonolactone, mevalonic acid, mevalonate, salts of mevalonic acid, mevalonolactone monohydrate, or any combination thereof.

More specifically, in one aspect the composition is a skin care composition for providing at least one skin care benefit in a subject, comprising an effective amount of an active ingredient selected from the group consisting of mevalonolactone, mevalonic acid, mevalonate, salts of mevalonic acid, mevalonolactone monohydrate, and any one combination thereof, wherein said composition provides at least one skin care benefit to a skin of said subject.

In one embodiment, the composition is a skin care composition for providing at least one skin care benefit in a subject, comprising one or more dermatologically or cosmetically acceptable components and an effective amount of an active ingredient selected from the group consisting of mevalonolactone, mevalonic acid, mevalonate, salts of mevalonic acid, mevalonolactone monohydrate, and any one combination thereof, wherein said composition provides at least one skin care benefit to a skin of said subject.

In one embodiment, the method is a method for providing at least one skin care benefit in a subject. comprising contacting a skin of said subject with a skin care composition comprising an active ingredient selected from the group consisting of mevalonolactone, mevalonic acid, mevalonolactone monohydrate, and any one combination thereof.

In one aspect, the at least one skin care benefit is selected from the group consisting of skin moisturizing, skin exfoliation (also referred to as skin peeling, desquamation, skin shedding), skin resurfacing, skin regeneration, skin renewal, improving epidermal cell turnover, preventing or retarding the appearance of the signs of aging of the skin (anti-aging). reducing the appearance of skin wrinkles, skin rejuvenation, strengthening the skin barrier function, and any one combination thereof.

Also provided are compositions and methods for recovering organic acids and salts and lactones thereof from aqueous solutions using water solvent crystallization. In one aspect, crystalline mevalonolactone*monohydrate (MVL*H₂O) and methods to crystalize mevalonolactone*monohydrate (MVL*H₂O) are disclosed

More specifically, in one aspect a method is provided for recovering a salt of mevalonate (X-MVA) from solutions containing the same. In another a method is provided for recovering mevalonolactone (MVL) from solutions containing a salt of mevalonate (X-MVA). mevalonolactone (MVL), mevalonoladone*monohydrate (MVL*H₂O), or combinations thereof. The whole process for the recovery of salts of mevalonate and/or recovery of mevalonolactone may preferably be carried out in an aqueous solution without the use of organic solvents.

The methods described herein can result in a high purity MVL product (>90% purity) that has a water like look with little or no color formation. As such, the MVL produced by methods described herein, can be scaled for large scale manufacture and are of particular interest for the personal care industry, where a highly purified MVL product is of greater value if it is water clear, with little or no trace of colored contaminates present.

This detailed description is intended only to acquaint others skilled in the art with Applicant's invention, its principles, and its practical application so that others skilled in the art may adapt and apply the invention in its numerous forms, as they may be best suited to the requirements of a particular use. This detailed description and its specific examples, while indicating certain embodiments, are intended for purposes of illustration only. This specification, therefore, is not limited to the described embodiments, and may be variously modified.

The present document is organized into a number of sections for ease of reading; however, the reader will appreciate that statements made in one section may apply to other sections. In this manner, the headings used for different sections of the disclosure should not be construed as limiting.

The headings provided herein are not limitations of the various aspects or embodiments of the present compositions and methods which can be had by reference to the specification as a whole. Accordingly, the terms defined immediately below are more fully defined by reference to the specification as a whole.

Unless defined otherwise, all technical and scientific terms used herein have the same meaning as commonly understood by one of ordinary skill in the art to which the present compositions and methods belongs. Although any methods and materials similar or equivalent to those described herein can also be used in the practice or testing of the present compositions and methods, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

### Definitions

In the specification and throughout the examples and the claims, the following definitions have been used:

The terms “Mevalonic acid (MVA)” or “(R)-Mevalonic acid” are used interchangeably herein and refer to (3R)-3,5-Dihydroxy-3-methylpentanoic acid with the chemical formula of CM-11204 and molar mass 148.16 g/mol. The carboxylate anion of mevalonic acid, is known as mevalonate. Mevalonic acid as described herein is a biologically active R-enatiomer.

The terms “salt of mevalonate” or “mevalonate salt” or “X-MVA” are used interchangeably herein and refer to a salt of mevalonic acid wherein X is the cation and MVA the carboxylate anion of mevalonic acid (hence X-MVA). The salt of mevalonate can be selected from the group consisting of Na-mevalonate (Na-MVA), K-mevalonate (K-MVA), ammonium mevalonate (NH₄-MVA), Lithium mevalonate (Li-MVA) any other monovalent salts of mevalonic acid, or any one combination thereof.

The terms “MevalonolaCone” or “(R)-Mevalonolactone” or “MVL” are used interchangeably herein and refer to (R)-3-Hydroxy-3-methyl-δ-valerolactone.

Aqueous solutions described herein include solutions containing both organic acids and their lactones which are converting to each other towards equilibrium concentrations. For example, mevalonic acid and mevalonolactone tends to equilibrium with each other. In this description, solutions containing organic acids do also contain lactone forms if not otherwise stated.

Mevalonolactone monohydrate is also referred to as MVL*H₂O.

SAC refers to a strong acid cation exchange resin.

WAC refers to a weak acid cation exchange resin.

DS refers to a dry substance content expressed as % by weight. Dry substance can be measured by Karl Fischer water titration.

RDS refers to a refractometric dry substance content, expressed as % by weight according to the correlation between refractometric index of aqueous sugar solution and DS.

IX or IEX refer to ion exchange process

BV/h refers to the volume flow rate through an ion exchange material contained in a column or operating unit. BV refers to bed volume which is volume of ion exchange material of specified ionic form contained in a column or operating unit.

Purity refers to the content of a component (such as Na-MVA, MVL*H₂O) on DS or RDS. The Area % calculation procedure reports the area of each peak in the chromatogram (such as an HPLC chromatogram) as a percentage of the total area of all peaks. For example, a purity of mevalonolactone of at least 90%, refers to the percentage being the peak area corresponding to MVL (90 in this case) relative to the total area of peaks (100) using chromatographic analysis. Purity can be measured by using HPLC (Rezex ROA-Organic Acid H+ (8%) column).

HPLC refers to high performance liquid chromatography.

Sodium mevalonate purity refers to the amount of sodium mevalonate as if all mevalonic acid, mevalonate, and mevalonolactone were in sodium mevalonate form divided by the total amount of dry substance.

Mevalonolactone purity refers to the total amount of mevalonolactone as if all mevalonic acid, mevalonate, and mevalonolactone were in mevalonolactone form divided by the total amount of dry substance.

Mevalonate yield refers to the amount of mevalonate in the target fraction (such as a nanofiltration permeate or a centrifugation cake) divided by the amount of mevalonate in the feed fraction (such as a nanofiltration feed or a centrifugation feed) as if all mevalonic acid, mevalonate, and mevalonolactone were in mevalonate form.

Mevalonolactone yield refers to the amount of mevalonolactone in the target fraction (such as a cation exchange product or a centrifugation cake) divided by the amount of mevalonolactone in the feed fraction (such as a cation exchange feed or a centrifugation feed) as if all mevalonic acid, mevalonate, and mevalonolactone were in mevalonolactone form.

Color refers to a color value under the International Commission for Uniform Process of Sugar Analysis (“ICUMSA”) sugar color grading system.

DSC thermogram was measured by using Mettler Toledo DSC822e differential scanning calorimeter. The measurement was run in standard 40 μL aluminum crucible in flowing nitrogen atmosphere with a flow rate of 80 mL/min. The temperature range was 0-50° C. and the heating rate was 2° C./min.

Optical rotation was measured from water solution which mevalonolactone concentration was 2 g/100 mL at a temperature of 20° C. by using Anton Paar MCP 300 Sucromet with 100 mm cuvette and Na 589 light.

The meaning of abbreviations is as follows: “sec” means second(s), “min” means minute(s), “h” or “hr.” means hour(s), “d” means day(s), “μL” means microliter(s), “mL” means milliliter(s), “L” means liter(s), “μM” means micromolar, means micrometer, “mM” means millimolar, “M” means molar, “mmol” means rnillimole(s), “μmole” mean micromole(s), “kg” means kilogram(s), “g” means gram(s), “μg” means microgram(s), “ng” means nanogram(s), “U” means unit(s), “bp” means base pair(s) and “kb” means kilobase(s).

### Starting Materials.

The solutions used in described methods comprise aqueous solutions comprising organic acids or salts or lactones thereof. More specifically, in one aspect the starting material is an aqueous solution comprising at least MVA and/or one salt of mevalonate (X-MVA). In one aspect the starting material is an aqueous solution comprising mevalonolactone (MVL) or mevalonolactone monohydrate (MVL*H₂O). The starting material may be selected for example from liquor originating from fermentation and/or from water solutions containing MVA, a salt of mevalonate, mevalonolactone, mevalonolactone monohydrate, or any one combination thereof.

In some embodiments, the aqueous solution comprises (or is derived in whole or in part from) a product of a fermentation. In some such embodiments, the aqueous solution is (or derived in whole or in part from) the product of a fermentation used to make the MVA, X-MVA and/or MVL to be purified. In some embodiments, the fermentation comprises culturing, in an aqueous culture medium comprising a carbohydrate, a recombinant microorganism comprising at least one recombinant polynucleotide sequence encoding an enzyme (or enzymes) capable of producing MVA, X-MVA and/or an MVL. The product of the fermentation process may be referred to as a fermentation “product” or “broth.” The product typically comprises many ingredients in addition to the X-MVA and/or an MVL to be purified, including, for example, monovalent and divalent salts, sugars, oligosaccharides, monosaccharides, amino acids, polypeptides, proteins, organic acids, nucleic acids, etc. The aqueous solutions can include some alcohol or other solvents from fermentation.

Examples of enzymes often useful for production of MVA include MvaE (Acetyl-CoA acetyltansferase/HMG-CoA reductase) and MvaS (Hydroxymethylglutaryl-CoA synthase). The mvaE gene encodes a polypeptide (MvaE) that possesses both thiolase and HMG-CoA reductase activities. The mvaS gene encode a polypeptide (MvaS) having HMG-CoA synthase activity. The enzymes capable of producing MVA (and corresponding nucleotide sequences) may originate from, but are not limited to, Listeria grayi, Enterococcus faecalis (Streptococcus faecalis), Enterococcus faecium, Enterococcus gallinarum, and Enterococcus casseliflavus.

The fermentation broth can be a broth obtained from a fermentation of any organism that is capable of producing MVA or MVL. In some embodiments, the fermentation broth is a broth obtained from an Escherichia coli fermentation. In some aspects, the starting aqueous solution can be a fermentation broth that neutral in pH, such as but not limiting to fermentation broths obtained from fermentations with Escherichia coli, wherein said fermentation broth comprises the mevalonic acid as a salt. The starting aqueous solution can also be a fermentation broth that is low in pH (pH 3-5) wherein said fermentation broth comprises the mevalonic acid partly in acid form and partly as a salt of mevalonate. The fermentation broth can be a clarified fermentation broth, wherein the clarification is obtained by ultrafiltration of the feed fermentation broth.

In some embodiments, the organic acid to be purified is MVA, and the MVA starting solution comprises (or is derived in whole or in part from) a product of a fermentation process wherein the fermentation process comprises culturing, in an aqueous culture medium, a recombinant microorganism comprising a recombinant polynucleotide sequence encoding an MvaE and a MvaS.

The fermentation broth can be clarified by removing biomass and any insoluble solids from said fermentation broth by at least one of precoat filtration, microfiltration, centrifugation, or ultrafiltration. Ultrafiltration can be beneficial to, for example, remove large biomolecules, such as endotoxins, proteins, nucleic acids and lipopolysaccharides.

Cell biomass may be separated, from a fermentation product using, for example, filtration, centrifugation, sedimentation and/or other process suitable for removing cell biomass.

### Nanofiltration

As described herein, in one aspect the object of the invention is a purification method for producing a crystalline form of a salt of mevalonic acid (also referred to as X-MVA or X-mevalonate) from an aqueous solution, comprising subjecting the aqueous solution comprising said salt of mevalonic acid (X-MVA) to a nanofiltration to produce a permeate and crystallizing said salt of mevalonic acid from said permeate by water solvent crystallization (see also Examples 1-4).

Nanofiltration (NF) is a pressure-driven membrane filtration-based process. The nanofiltration provides two fractions: a retentate and a permeate. In one aspect of the invention, the NF process aims to purify the salt of mevalonic acid (such as but not limiting to a Na-mevalonate, K-mevalonate, Li-mevalonate, ammonium mevalonate or other monovalent salt of mevalonic acid) to the permeate (also referred to as the filtrate). The retentate (also referred to as the concentrate) from the process is waste containing antifoam, endotoxin, colour components, divalent salts and larger molecules. In one embodiment of the invention an aqueous solution is used as a feed for nanofiltration to obtain a permeate with a high content of a salt of mevalonate (at least 60% sodium mevalonate in permeate, Examples 1-4) and only small amount of waste materials.

The starting aqueous solution can be a fermentation broth that is neutral in pH, such as but not limiting to fermentation broths obtained from fermentations with Escherichia coli, wherein said fermentation broth comprises the mevalonic acid as a salt. The starting aqueous solution can also be a fermentation broth that is low in pH (pH 3-5) wherein said fermentation broth comprises the mevalonic acid partly in acid form and partly as a salt of mevalonate.

The nanofiltration in accordance with the present invention may be carried out as a batch process or a continuous process.

The nanofiltration is typically carried out at a temperature in the range of 5 to 80° C., preferably 30 to 75° C. and most preferably 50 to 70° C. The pressure in the nanofiltration is typically in the range of 5 to 60 bar, preferably 10 to 50 bar and most preferably 20 to 45 bar. The pH may be in the range of 1 to 10, preferably 3 to 9 and most preferably 6 to 9. The pH depends on the composition of the starting solution and the membrane used for the nanofiltration and the stability of the components to be recovered. If necessary, the pH of the starting solution may be adjusted to the desired value before nanofiltration.

The nanofiltration is typically carried out with a flux of 1 to 100 l/m²h, preferably with a flux of 2 to 50 l/m²h, and most preferably with a flux of 3 to 12 l/m²h depending on the concentration and the viscosity of the nanofiltration feed.

The nanofiltration membrane used in the present invention can be selected from polymeric and inorganic membranes having MgSO₄ retention of 50 to 99% (at 25° C., 2g/l concentration, 8 bar, pH 6), preferably 70 to 99% (at 25° C., 2 g/l concentration, 8 bar, pH 6), more preferably 80 to 98% (at 25° C., 2 g/l concentration, 8 bar, pH 6), most preferably 90 to 98% (at 25° C., 2 g/l concentration, 8 bar, pH 6). In one aspect the nanofiltration membrane is the nanofiltration membrane XN45 that has a MgSO₄ retention of about 92-98%.

Nanofiltration membranes having MgSO₄ retention of 99% or greater MgSO4 retention have too high monovalent salt retention and low MWCO and thus too high X-MVA salt retention. Membranes having MgSO₄ retention of <90% MgSO4 retention don't give as high purification as also small divalent salts are passing.

In some embodiments, the membrane has a molecular weight cut-off (MWCO) in the range of about 100 to about 700 Daltons MWCO membranes, such as but not limiting to the TriSep XN45 membrane. The TriSep XN45 membrane is characterized as a 300-500 Dalton MWCO membrane. In some embodiments, the membrane has a molecular weight cut-off (MWCO) in the range of about 150 to about 400 Daltons. In some embodiments, the membrane has a molecular weight cut-off (MWCO) in the range of about 150 to about 300 Daltons, such as but not limiting to Suez duratherrn EXL DL and DK membranes. Suez duratherm EXL DL and DK membranes are characterized as 150-300 Dalton MWCO membranes. MWCO and MgSo4 retention are both important parameters for selecting nanofiltration membranes. In one aspect the membranes for the process as described are nanofiltration membranes having a MWCO above 150 Da, but also high divalent salt rejection (>90% MgSO4)), such as but not limiting to TriSep XN45 type membranes.

In some embodiments, the membrane has a molecular weight cut-off (MWCO) in the range of about 100 to about 900 Daltons and a MgSO₄ retention of about 50 to 99% at 25° C. In some embodiments, the membrane has a molecular weight cut-off (MWCO) in the range of about 150 to about 500 Daltons and a MgSO₄ retention of about 80-99% at 25° C. In some embodiments, the membrane has a molecular weight cut-off (MWCO) in the range of about 150 to about 300 Daltons and a MgSO₄ retention of about 98 to 99% at 25° C.

The nanofiltration membranes which are useful in the present invention may have a negative or positive charge. The membranes may be ionic membranes, i.e. they may contain cationic or anionic groups. but even neutral membranes are useful. The nanofiltration membranes may be selected from hydrophobic and hydrophilic membranes.

The typical form of nanofiltration membranes comprises spiral wound membranes. The membrane configuration may also be selected e.g. from flat sheets, tubes and hollow fibers. “High shear” membranes, such as vibrating membranes and rotating membranes can also be used. The membrane can be tubular, spiral or flat in shape.

The nanofiltration equipment useful in the present invention comprises at least one nanofiltration membrane element dividing the starting material (feed) into a retentate and permeate section. The nanofiltration equipment typically also include means for controlling the pressure and flow, such as pumps and valves and flow and pressure meters and controllers. The equipment may also include several nanofiltration membrane elements in one pressure vessel in different combinations, arranged in parallel or in series.

The yield of X-MVA (the salt of mevalonate, such as for example Na-mevalonate) in the nanofiltration is typically more than 70%, preferably more than 80% and most preferably more than 90% on the Na-mevalonate present in the starting material.

Na-mevalonate content in the permeate is more than 50 on DS, preferably more than 60% on DS, more preferably more than 70% on DS, and most preferably more than 80% on DS.

The nanofiltration permeate may be subjected to further concentration, by evaporation or any means know in the art to further concentrate the salt of mevalonate, such as but not limiting to evaporation under reduced pressure (vacuum evaporation) to produce a concentrated aqueous syrup. Na-mevalonate content in concentrated aqueous syrups can be more than 60% on DS, preferably between 65%-95% on DS.

The nanofiltration permeate may be subjected to further purification steps selected from ion exchange, evaporation, electrodialysis and filtration. These further purification steps may be carried out before or after said membrane filtration. Furthermore, the recovered salt of mevalonate fractions may be subjected to one or more further steps, such as evaporation, concentration, filtration ion exchange, active carbon treatment, sterile filtration, crystallization, intermediate crystallization, nanofiltration and chromatographic fractionation. The recovered salt of mevalonate fraction(s) may be treated in different ways, depending on the purity of the fractions. In some embodiments, the permeate collected from the nanofiltration and/or the concentrated aqueous syrup is subjected to a subsequent crystallization step.

In one aspect, as described herein, the method is a method for producing a crystalline form of a salt of mevalonic acid (X-MVA) from an aqueous solution, comprising subjecting the aqueous solution comprising said salt of mevalonic acid to a purification step, whereby said purification step produces a purified solution comprising X-MVA of a purity of at least 60%, and crystallizing said salt of mevalonic acid from said purified solution by water solvent crystallization, wherein said purification step comprises subjecting the aqueous solution comprising said salt of mevalonic acid to a nanofiltration to produce a permeate, wherein said permeate comprises X-MVA at a purity of at least 60%.

Alternatively, in one aspect, the method is a method for producing a crystalline form of a salt of mevalonic acid (X-MVA) from an aqueous solution, comprising subjecting the aqueous solution comprising said salt of mevalonic acid to a purification step, whereby said purification step produces a purified solution comprising X-MVA of a purity of at least 60%, and crystallizing said salt of mevalonic add from said purified solution by water solvent crystallization, wherein said purification step comprises subjecting the aqueous solution comprising said salt of mevalonic acid to microfiltration, electrodialysis, ion exchange, filtration, active carbon treatment, evaporation, concentration, sterile filtration, chromatographic fractionation, or any one combination thereof.

### Cation Exchange

In one aspect, the specification provides a method for producing a water solubilized mevalonolactone from an aqueous solution wherein the method comprises: a) producing a crystalline form of a salt of mevalonic acid from an aqueous solution by subjecting the aqueous solution comprising said salt of mevalonic acid (X-MVA) to a nanofiltration to produce a permeate and crystallizing said salt of mevalonic acid from said permeate by water solvent crystallization to produce crystals of said salt of mevalonic acid; and, b) dissolving the crystals of (a) in water to produce a water solubilized salt of mevalonic acid and subjecting said liquid to cation exchange thereby converting said water solubilized salt of mevalonic acid to water solubilized mevalonolactone (Example 9-12).

In another aspect, the specification provides a method for producing mevalonolactone from an aqueous solution comprising a salt of mevalonic acid salt (X-MVA)), comprising subjecting the aqueous solution comprising said salt of mevalonate to cation exchange thereby converting said aqueous solution comprising a salt of mevalonate to an aqueous solution comprising mevalonolactone (MVL).

### Strong Acid Cation Exchange Resins (SAC Resins)

The SAC resins may have a styrene or acrylic skeleton. In one embodiment of the invention, the resin is a sulphonated polystyrene-co-divinylbenzene resin. Other alkenyl aromatic polymer resins like those based on monomers like alkyl-substituted styrene or mixtures thereof can also be applied. The resin may also be crosslinked with other suitable aromatic crosslinking monomers, such as divinyltoluene, divinylxylene, divinylnaphtalene, divinylbenzene, or with aliphatic crosslinking monomers, such as isoprene, ethylene glycol diacrylate, ethylene glycol dimethacrylate, N,N′-methylene bis-acrylamide or mixtures thereof. The cross-linking degree of the resin is typically from about 1 to about 20%, preferably from about 3 to about 8% of the cross-linking agent, such as divinyl benzene (DVB), The SAC resins used for the ion exchange of the invention may be in a multivalent, divalent or monovalent cation form. The monovalent cation forms may be selected from H⁺, Na⁺ and K⁺, for example. Examples of divalent cation forms are Ca²⁺, Mg²⁺, Zn²⁺, Sr²⁺ and Ba²⁺. An example of a trivalent cation form is Al³⁺. In a preferred embodiment of the invention, the SAC resin used for the ion exchange of the invention is in a monovalent H⁺ form. A typical mean average particle size of the resin is 10 to 2000 μm, preferably 300 to 1200 μm.

In one embodiment, a monovalent SAC resin is used for the ion exchange of a salt of mevalonic acid (X-MVA) to produce mevalonolactone (MVL), wherein the monovalent cation is in a Ht form.

Weak Acid Cation Exchange Resins (SAC Resins)

The WAC resins are acrylic cation exchange resins, having carboxylic functional groups. The acrylic WAC resin is typically derived from the group consisting of an acrylate ester, acrylonitrile, acrylic acids and mixtures thereof. The acrylate ester is selected from the group consisting of methyl methacrylate, methyl acrylate, ethyl acrylate and butyl acrylate. The matrix of the WAC resins may also be other than acrylic. The active functional groups of the WAC resins may also be other than carboxylic groups. They may be selected from other weak acids, for example. The WAC resin may be in a H⁺, Na⁺, K⁺, Ca²⁺ or Mg²⁺ form, preferably in a H⁺ or Na⁺ form. Other ion forms may also be used.

The WAC resin is crosslinked with an aromatic crosslinker, preferably divinylbenzene (DVB). It may also be crosslinked with an aliphatic crosslinker, such as isoprene, 1,7-octadiene, trivinylcyclohexane, diethylene glycol divinylether. The crosslinking degree is from 1 to 20%, preferably from 3 to about 8% DVB. The average particle size of the WAC resin is from 10 to 2000 μm, preferably from 300 to 1200 μm.

The ion-exchange is preferably performed by using cation exchange resin, particularly strong acid cation resin, particularly in H⁺-ion form.

In one embodiment, a monovalent WAC resin is used for the ion exchange of a salt of mevalonic acid (X-MVA) to produce mevalonolactone (MVL), wherein the monovalent cation is in a H⁺ form.

### Water Solvent Crystallization

As described herein, in one aspect the object of the invention is a method for producing a crystalline form of a salt of mevalonic acid (also referred to as X-MVA or X-mevalonate) from an aqueous solution, comprising subjecting the aqueous solution comprising said salt of mevalonic acid (X-MVA) to a purification step such as but not limiting to a nanofiltration to produce a high purity solution (permeate) that allows for crystallization of said X-MVA, and crystallizing said salt of mevalonic acid from said permeate by water solvent crystallization (Examples 5-8).

In another aspect of the invention, the specification provides a method for producing mevalonolactone from an aqueous solution comprising a salt of mevalonic acid salt (X-MVA)), comprising subjecting the aqueous solution comprising said salt of mevalonate to cation exchange thereby converting said aqueous solution comprising a salt of mevalonate to an aqueous solution comprising mevalonolactone (MVL) and optionally followed by concentrating said MVL solution and crystallization said solution to obtain MVL*monohydrate crystals (Examples 13-20)

The term “water solvent crystallization” refers to a crystallization that is carried out using an aqueous solvent such as water, without the use of any organic solvent.

### Water Solvent Crystallization of X-MVA

The crystallization of X-MVA may be carried out by traditional methods, such as cooling crystallization or precipitation crystallization in a temperature range of 10 to 80° C. The crystallization of X-MVA may also advantageously be carried out by a boiling crystallization method or by a boiling and cooling crystallization method. In one embodiment of the disclosure, the crystallization of X-MVA is carried out from a solution (feed solution) having an X-MVA purity of more than 60%, preferably more than 70%, more preferably more than 80%, most preferably more than 90% and especially more than 95% on DS. The crystallization typically provides a crystalline X-MVA product having a purity of more than 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 89%, 90%,91%, 92%, 93%, 94% preferably more than 95% and most preferably more than 99%, on DS.

The solution containing X-MVA can be first evaporated to an appropriate dry substance content (e.g. to an DS of about 60 to 90%) depending on the X-MVA content of the solution. The syrup DS concentration must be high enough to have crystallization potential (supersaturation). The minimum DS content depends on the temperature and purity of the syrup. The higher the purity of the syrup is, the lower the syrup DS needs to be. If the DS is too high, then the crystallization is retarded or the crystal suspension come too dense for efficient crystal separation. In one embodiment of the invention the syrup containing Na-MVA has a purity between 65-99%.

The supersaturated solution may be seeded with seed crystals of X-MVA. The seeds, if used, are crystals in a dry form or they are suspended in a solvent, preferably in water, and preferably the crystal size is reduced for example by pulverizing or milling. Temperature of evaporation of solution containing X-MVA may range between 30° C. to 80° C. After seeding, the crystallization mass is subjected to cooling with simultaneous mixing until the crystallization yield and viscosity is optimal for the separation of crystals. The cooling time is preferably 10 to 60 hours. The temperature drop during cooling is preferably 5 to 40° C. The seeded syrup comprising Na-MVA can be cooled to a temperature of about 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C. to about 75° C. or higher. The cooling can occur for a period of about 2 hr., 3 hr., 4 hr., 5 hr., 6 hr., 7 hr., 8 hr., 9 hr., 10 hr., 11 hr., 12 hr., 13 hr., 14 hr., 15 hr., 16 hr., 17 hr., 18 hr. or more. The cooling can occur under continuous stirring or mixing. Mixing can be beneficial in controlled crystallization. Mixing prevents crystals from settling, maintain a crystal growth and reduces spontaneous crystal formation. Mixing is also promoting beneficial heat and mass transfer. The crystallization mass may then be mixed at the final temperature for a period of time, preferably 0.5 to 24 hours, to reach the maximum crystallization yield. The crystals are separated from the mother liquor for example by filtration or centrifugation.

In one embodiment of the present invention, X-MVA crystals having a high purity are X-MVA crystals having a content over 97% on DS, preferably over 98% on DS, and more preferably over 99% on DS that are obtained by one crystallization step (=single-stage crystallization) from a solution having X-MVA content over 65% on DS without dissolving and recrystallization steps. Single stage crystallization may comprise boiling and cooling steps but no recrystallization step.

In another embodiment of the invention, the crystallization of X-MVA comprises washing as a further step. The washing is typically made in connection with the crystal separation from the mother liquor. Additional washing can be made by mixing aqueous washing solvent and crystal cake and separating crystals thereafter. The washing solvent can be water. This embodiment of the invention typically provides X-MVA with a purity of more than 98%.

Seed crystals can be made by various processes. In some embodiments, the dry seeds are milled to get smaller particle size. The desired amount of seed crystals may depend on. for example. the size of the seed crystals. In some embodiments, crystallization is initiated without adding seed crystals to the supersaturated solution. In some such embodiment, for example, seeding is affected using spontaneous seeding.

In some embodiments, initiation of crystallization (e.g., addition of seed crystals) is carried out when the dry solids content of the syrup is at least about 60% (by weight). In some embodiments, initiation of crystallization (e.g., addition of seed crystals) is carried out when the dry solids content of the syrup is at least about 70% (by weight). In some embodiments, initiation of crystallization (e.g., addition of seed crystals) is carried out when the dry solids content of the syrup is at least about 80% (by weight). In some embodiments, initiation of crystallization (e.g., addition of seed crystals) is carried out when the dry solids content of the syrup is from about 60 to about 90% (by weight). In some embodiments, initiation of crystallization (e.g., addition of seed crystals) is carried out when the dry solids content of the syrup is from about 70 to about 90% (by weight). In some embodiments, initiation of crystallization (e.g., addition of seed crystals) is carried out when the dry solids content of the syrup is from about 80 to about 90% (by weight). In some embodiments, initiation of crystallization (e.g., addition of seed crystals) is carried out when the dry solids content of the syrup is from about 80 to about 88% (by weight).

The evaporation can be continued after seeding, if the crystal growth potential and viscosity allows. After evaporation, the crystallization mass is subjecting to cooling with simultaneous mixing, until the crystal content and viscosity is optimal for the separation of crystals. The crystallization mass is typically cooled to a temperature of 10 to 75° C. The crystallization mass may then be mixed at the final temperature for a period of time, preferably from 0.5 hours to 24 hours to reach the maximum crystallization yield, where after the crystals are separated for example by filtering or centrifuging. The process of the invention typically comprises washing of the crystals as a further step. The washing is typically made in connection with the crystal separation from the mother liquor. Additional washing can be made by mixing washing solvent and crystal cake and separating crystals thereafter. The washing solvent can be water.

In some embodiments, recrystallization is performed one or more times to increase X-MVA purity. Recrystallization may be carried out by, for example, dissolving the X-MVA crystals in water (typically deionized water), bringing the resulting solution to a supersaturated state with respect to X-MVA (via, for example, evaporation), seeding and crystallizing using the crystallization-by-cooling methodology described above.

In some embodiments, yield is increased by performing crystallization of the mother liquor produced by the initial crystallization. Such a crystallization may be carried out by, for example, bringing the mother liquor to a supersaturated state with respect to X-MVA (via, for example, evaporation), seeding and crystallizing using the crystallization-by-cooling methodology described above.

The crystallization described in this specification does not require an organic solvent to be present in the solution. Crystalline X-MVA which is free from organic solvents is obtained. Crystalline X-MVA which has been produced without adding any organic solvents in crystallization steps is essentially free from organic solvents.

Although organic solvents can be added to aqueous solutions to modify crystallization and crystal separation performance, such addition of organic solvents has disadvantages such as, but not limiting to, obtaining crystals or purified products that comprise small amounts of these organic solvents, which are not desired in cornmercial compositions such as personal care composition.

### Water Solvent Crystallization of MVL*monohydrate

As described herein, it has been found surprisingly and unexpectedly that when a high purity syrup of MVL was prepared by methods described herein and cooled at temperatures below 23° C. for several weeks mevalonolactone monohydrate (MVL*H₂O) crystals formed spontaneously. These mevalonolactone monohydrate (MVL*H₂O) crystals can be used as seed material for further facilitating, crystallization of MVL*H₂O as described herein. MVL*H₂O crystallization was accomplished at a temperature below the melting point, including at temperatures below 23° C., 22° C., 21° C., 26° C. 19° C., 18° C., 17° C., 16° C., 15° C. 14° C., 13° C., 12° C., 11° C., 10° C., 9° C., 8° C., 7° C., 6° C., 5° C., 4° C., 3° C., 2° C., 1° C. and down to 0° C. Crystallization of MVL*H₂O is possible below 0° C. until down to the freezing point of the solution. In one embodiment of the invention, the crystallization of MVL*monohydrate (MVL* H₂O) is carried out from an aqueous solution having an MVL purity of more than 55%, preferably more than 70%, more preferably more than 80%, most preferably more than 90% and especially more than 95% on DS. The crystallization typically provides a crystalline MVL*H₂O product having a purity of more than 65%, 66%, 67%, 68%. 69%, 70%, 71%, 72%, 73%, 74%, 75%, 76%, 77%, 78%, 79%, 80%, 81%, 82%, 83%, 84%, 85%, 86%, 87%, 89%, 90%,91%, 92%, 93%, 94% preferably more than 95% and most preferably more than 99%, on DS.

In one aspect cooling crystallization of MVL* H₂O is preferred over constant temperature crystallization due to practical reasons. Constant temperature crystallization requires very high seeding supersaturation which results in less controllable process than with cooling. MVL*H2O liberates lot of heat during crystallization and that heat must be removed from the system to reach desirable crystallization yields. In some instance, like having very high seeding supersaturation, the crystallization is fast and the temperature of the crystallizing suspension can rise significantly. This can be overcome by having effective cooling means or utilizing lower seeding supersaturation and controlled cooling.

The solution containing MVL can be first evaporated to an appropriate dry substance content (e.g. to a DS of about 65 to 90%) depending on the MVL content of the solution. The syrup DS concentration must be high enough to have crystallization potential (supersaturation). The minimum DS content depends on the temperature and purity of the syrup. The higher the purity of the syrup is, the lower the syrup DS needs to be. If the DS is too high, then the crystallization is retarded or the crystal suspension come too dense for efficient crystal separation. The supersaturated solution may be seeded with seed crystals of MVL*H₂O crystals. The seeds, if used, are crystals in a dry form or they are suspended in a solvent, preferably in water and preferably the crystal size is reduced for example by pulverizing or milling. After seeding, the crystallization mass is subjected to cooling with simultaneous mixing until the crystallization yield and viscosity is optimal for the separation of crystals.

Conditions for crystallizing MVL*H₂O are further described in Examples 13-20.

In one aspect the mevalonolactonemonohydrate (MVL*H₂O) crystals can be warmed up to a temperature such that the crystals turn into a liquid (such as but not limiting to room temperatures of about 23° C.-30° C.) without adding any additional water. Such a liquid comprises about 87% MVL and 13% water.

Mevalonolactone monohydrate (MVL*H₂O) crystals can be dissolved in water to produce highly purified MVL (>90% purity) that has a water like look and viscosity with little or no color. This highly purified MVL solution can be further concentrated by evaporation while retaining a water like look.

Compositions Comprising MVA, X-MVA, MVL and/or MVL*H₂O

The crystalline X-MVA or a composition comprising the crystalline X-MVA may be used as an ingredient for example for dietary supplements, infant and human nutrition, pharmaceuticals and cosmetics.

The MVA, X-MVA, MVL and/or MVL*H₂O purified by a process of this specification or a composition comprising the MVA, X-MVA, MVL and/or MVL*H₂O described herein can be used as an ingredient for example for dietary supplements, personal care compositions (such as but not limiting to skin care, oral care, hair care compositions), pharmaceuticals and cosmetics.

For the purpose of using MVA or MVL in the personal care industry, the purified product is of greater value if it is water clear, with little or no trace of colored contaminates present. As described herein the highly purified MVL product resulting from the methods described herein had a water clear look with little or no remaining color.

### Compositions for Topical Applications (Skin Care Compositions)

Also provided by the present invention are mevalonolactone, mevalonic acid mevalonate, salts of mevalonic acid, and mevalonolactone monohydrate obtained by the methods of the invention and compositions for topical application for skin care, skin supplement, hair care, and oral care, comprising said mevalonolactone, mevalonic acid, mevalonate, salts of mevalonic acid, mevalonolactone monohydrate, or any combination thereof.

The term “topical application” as used herein refers to external application to the skin, mucous membranes, hair, or scalp.

As used herein, the terms “skin care composition” and “composition for topical application” are used interchangeably herein and refer to compositions comprising at least one skin care benefit agent (active ingredient) selected from the group consisting of mevalonolactone, mevalonic acid, mevalonate, salts of mevalonic acid, mevalonolactone monohydrate, and any one combination thereof, capable of providing a benefit for skin care, skin supplement, hair care, and oral care.

In one aspect, the term “skin care composition” or “compositions for topical application” includes compositions comprising an effective amount of at least one skin care benefit agent (active ingredient) selected from the group consisting of mevalonolactone, mevalonic acid, mevalonate, salts of mevalonic acid mevalonolactone monohydrate, and any one combination thereof, capable of providing a skin care benefit.

As used herein the term “skin care benefit” refers to a benefit provided by the composition for topical application (skin care composition) comprising an effective amount of a skin care benefit agent (active ingredient) selected from the group consisting of mevalonolactone, mevalonic acid, mevalonate, salts of mevalonic acid, mevalonolactone monohydrate, and any one combination thereof, when applied topically to a skin, mucous membranes, hair, or scalp, wherein the benefit is selected from the group consisting of skin moisturizing (maintaining or increasing the water content of the epidermis, and, protecting the skin against dehydration by maintaining, restoring and/or strengthening the skin barrier function), skin exfoliation (also referred to as skin peeling, desquamation, skin shedding), skin resurfacing, skin regeneration, skin renewal, improving epidermal cell turnover, preventing or retarding the appearance of the signs of aging of the skin, reducing the appearance of skin wrinkles, skin rejuvenation, strengthening the skin barrier function, and any one combination thereof.

An effective amount of an active ingredient selected from the group consisting of mevalonolactone, mevalonic acid, mevalonate, salts of mevalonic acid, mevalonolactone monohydrate, or any one combination thereof, in a composition refers to the amount of the active ingredient that helps provide a benefit for skin care, skin supplement, hair care, and oral care.

The term “compositions for topical application” or “composition for topical application” or “skin care composition” includes compositions in any form such as aqueous solutions, emulsions, serums, jellies, masks, patches, face masks, peel-off masks, lotions, topical moisturizers, creams, pastes, balms, ointments, pomades, gels, liquids, sprays, foam, kits, oral care, hair care, or any one combination thereof.

Skin care compositions can be contacted with (applied to, administered to) any surface of the skin, mucous membranes, hair, or scalp. Skin care compositions can be topically applied to the skin and are referred to herein as compositions for topical application.

As used herein “administer” or “administering” or “contacting” is meant the action of introducing one or more active ingredients (including the active ingredients selected from the group consisting of mevalonolactone, mevalonic acid, mevalonate, salts of mevalonic acid, mevalonolactone monohydrate, and any one combination thereof), or one or more skin care composition(s) to a skin of a subject.

As used herein “contacting a skin with a skin care composition”, “contacting a skin with a composition for topical application”, “administering to a skin a composition for topical application” and “administering to a skin a skin care composition” are used interchangeably, and refer to the action of introducing one or more active ingredient selected from the group consisting of mevalonolactone, mevalonic acid, mevalonate, salts of mevalonic acid, mevalonolactone monohydrate, and any one combination thereof to a skin, or introducing one or more compositions comprising said active ingredient to a skin of a subject.

Administering one or more active ingredient selected from the group consisting of mevalonolactone, mevalonic acid, mevalonate, salts of mevalonic acid, mevalonolactone monohydrate, and any one combination thereof, or introducing one or more compositions comprising said active ingredient to a skin of a subject in need thereof includes applying or introducing one or more active ingredient/composition comprising said active ingredient to a scalp, a skin surface, and to in-vitro or in-vivo skin cells.

As used herein, the terms “strengthening the skin barrier function”, “improved skin barrier function”, “increased skin barrier function”, “improved skin barrier” and “increased skin barrier” are used interchangeably herein and refer to skin barrier strengthening or increase in skin barrier or enhancement or improvement of skin barrier function indicated by the skin (such as, but not limiting to, its permeability barrier) being “tighter/stronger” and wherein the skin limits or avoids compounds from getting in (such as microbes, pollutants etc.) but also wherein the skin limits or avoids the amount of water that gets out, i.e. the skin barrier function gets improved.

The term “subject”, as used herein, means an animal having a skin that separates and defines the animal inside from the outside. Preferably, the subject is a mammal, including for example livestock (including, but not limited to, cattle, horses, pigs, and sheep), and humans, or birds, such as chickens.

In one embodiment the subject is a human.

In one embodiment the subject may be female.

In one embodiment the subject may be male.

In one embodiment the subject may be with a non-binary gender.

In one embodiment the subject is a healthy subject.

In one embodiment, the subject suffers from dry skin, skin infection or any other transient or chronic condition responsible for skin dehydration, skin redness, skin irritation, skin aging or skin wrinkles.

In one aspect, the effective amount of an active ingredient selected from the group consisting of mevalonolactone, mevalonic acid, mevalonate, salts of mevalonic acid, mevalonolactone monohydrate, and any one combination thereof, in a composition refers to the amount of the active ingredient that helps provide a skin care benefit, such as but not limiting to skin moisturization, skin exfoliation (also referred to as skin peeling, desquamation, skin shedding), skin resurfacing, skin regeneration, skin renewal, improving epidermal cell turnover, preventing or retarding the appearance of the signs of aging of the skin, reducing the appearance of skin wrinkles, skin rejuvenation, or strengthening the skin barrier function.

In one aspect, the effective amount of an active ingredient selected from the group consisting of mevalonolactone, mevalonic acid, mevalonate, salts of mevalonic acid, mevalonolactone monohydrate, and any one combination thereof, in a composition for topical application is between 0.01% to 10.0% active ingredient on a weight basis relative to a total weight of said composition.

In one aspect, the effective amount of an active ingredient selected from the group consisting of mevalonolactone, mevalonic acid, mevalonate, salts of mevalonic acid, mevalonolactone monohydrate, and any one combination thereof, is at least about least 0.01%, 0.02%, 0.03%, 0.04%, 0.05%, 0.06%, 0.07%, 0.08%, 0.09%, 0.1%, 0.2%, 0.3%, 0.5%, 0.6%. 0.7%, 0.8%, 0.9%. 1%, 2%, 3%, 4%, 5%, 6%, 7%, 8%, 9% up to 10% on a weight basis relative to a total weight of said composition.

In one embodiment, the composition is a skin care composition for providing at least one skin care benefit in a subject, comprising an effective amount of an active ingredient selected from the group consisting of mevalonolactone, mevalonic acid, mevalonate, salts of mevalonic acid, mevalonolactone monohydrate, and any one combination thereof. wherein said composition provides at least one skin care benefit to a skin of said subject.

In one embodiment, the composition is a skin care composition for providing at least one skin care benefit in a subject, comprising one or more dermatologically or cosmetically acceptable components and an effective amount of an active ingredient selected from the group consisting of mevalonolactone, mevalonic acid, mevalonate, salts of mevalonic acid, mevalonolactone monohydrate, and any one combination thereof, wherein said composition provides at least one skin care benefit to a skin of said subject.

In one embodiment, the composition is a skin care composition for providing at least one skin care benefit in a subject, comprising an effective amount of mevalonolactone, wherein said composition provides at least one skin care benefit to said subject. Said composition can further comprises one or more dermatologically or cosmetically acceptable components.

In one embodiment, the composition is a skin care composition for strengthening the skin barrier function. Strengthening the skin barrier function is a synonym to an improvement of or an increase in the skin barrier function or enhancement or increase of the skin barrier.

In one embodiment, the composition is a skin care composition for providing skin moisturization (an increased skin water content).

In one embodiment, the composition is a skin care composition for providing skin exfoliation.

The dermatologically acceptable component can be a dermatologically acceptable carrier comprising about 10% to about 99% on a weight basis relative to a total weight of the skin care product.

Compositions for topical applications described herein may further comprise one or more dermatologically or cosmetically acceptable components known or otherwise effective for use skin care, provided that the optional components are physically and chemically compatible with the essential components described herein, or do not otherwise unduly impair product stability, aesthetics, or performance. Non-limiting examples of such optional components are disclosed in International Cosmetic Ingredient Dictionary, Ninth Edition, 2002, and CTFA Cosmetic Ingredient Handbook, Tenth Edition, 2004.

In one aspect, the dermatologically or cosmetically acceptable component is a dermatologically acceptable carrier comprising from about 10 wt. % to about 99.9 wt. %, alternatively from about 50 wt. % to about 95 wt. %, and alternatively from about 75 wt. % to about 95 wt. %, of a dermatologically acceptable carrier. Carriers suitable for use with the composition(s) may include, for example. those used in the formulation of mousses, tonics, gels, skin moisturizers and lotions. The carrier may comprise water; organic oils; silicones such as volatile silicones, amino or non-amino silicone gums or oils, and mixtures thereof; mineral oils; plant oils such as olive oil, castor oil, rapeseed oil, coconut oil. wheatgerm oil, sweet almond oil, avocado oil, macadamia oil, apricot oil, safflower oil, candlenut oil, false fax oil, tamanu oil, lemon oil and mixtures thereof; waxes; and organic compounds such as C2-C10 alkanes, acetone, methyl ethyl ketone, volatile organic C1-C12 alcohols, esters of C1-C20 acids and of C1-C8 alcohols such as methyl acetate, butyl acetate, ethyl acetate, and isopropyl myristate, dimethoxyethane, diethoxyethane, C10-C30 fatty alcohols such as lauryl alcohol, cetyl alcohol, stearyl alcohol, and behenyl alcohol; C13-C30 fatty adds such as lauric acid and stearic acid; C10-C30 fatty amides such as lauric diethanolamide; C10-C30 fatty alkyl esters such as C10-C30 fatty alkyl benzoates; hydroxypropylcellulose, and mixtures thereof. In one aspect, the carrier comprises water, fatty alcohols, volatile organic alcohols, and mixtures thereof. Other carriers can be formulated by those of ordinary skill in the art.

Compositions for topical applications described herein may further comprise from about 0.1% to about 10%, and alternatively from about 0.2% to about 5.0%, of a gelling agent to help provide the desired viscosity to the composition(s). Non-limiting examples of suitable optional gelling agents include crosslinked carboxylic acid polymers; unneutralized crosslinked carboxylic acid polymers; unneutralized modified crosslinked carboxylic acid polymers; crosslinked ethylene/maleic anhydride copolymers; unneutralized crosslinked ethyleneimaleic anhydride copolymers; unneutralized crosslinked alkyl ether/acrylate copolymers; unneutralized crosslinked copolymers of sodium polyacrylate, mineral oil, and PEG-1 trideceth-6; unneutralized crosslinked copolymers of methyl vinyl ether and maleic anhydride; hydrophobically modified nonionic cellulose polymers; hydrophobically modified ethoxylate urethane polymers; and combinations thereof. In this context, the term “unneutralized” means that the optional polymer and copolymer gelling agent materials contain unneutralized acid monomers.

The cosmetically acceptable medium may contain a fatty substance in a proportion generally of from about 10 to about 90% by weight relative to the total weight of the product. where the fatty phase containing at least one liquid, solid or semi-solid fatty substance. The fatty substance includes, but is not limited to, oils, waxes, gums, and so-called pasty fatty substances. Alternatively, the products may be in the form of a stable dispersion such as a water-in-oil or oil-in-water emulsion. Additionally, the skin care products may contain one or more conventional cosmetic or dermatological additives or adjuvants, including but not limited to, antioxidants, preserving agents, fillers, surfactants, UVA and/or UVB sunscreens, fragrances, thickeners, wetting agents and anionic, nonionic or amphoteric polymers, and dyes or pigments (colorant agents).

The dermatologically acceptable carrier may be a moisturizer formulation containing at least one emulsifier, or at least one surfactant, or any combination thereof.

Compositions for topical applications can further comprise skin care active ingredient materials including sun screen agents, moisturizers, humectants, benefiting agents skin, depositing agents such as surfactants, occlusive agents, moisture barriers, lubricants, emollients, anti-aging agents, antistatic agents, abrasive, antimicrobials, conditioners, exfoliants, fragrances, viscosifying agents, salts, lipids, phospholipids, vitamins, foam stabilizers, pH modifiers, preservatives, suspending agents, silicone oils, silicone derivatives, essential oils, oils, fats, fatty acids, fatty acid esters, fatty alcohols, waxes, polyols, hydrocarbons, and mixtures thereof.

Skin care compositions described herein can also be part of a kit for providing one or more skin care benefits such as, but not limiting to, a kit for increasing skin moisturization, a kit for increasing skin exfoliation or a kit for providing anti-aging.

In one aspect the kit is a kit comprising the at least one composition for topical application comprising an effective amount of an active ingredient selected from the group consisting of mevalonolactone, mevalonic acid, mevalonate, salts of mevalonic acid, mevalonolactone monohydrate, and any one combination thereof, wherein said composition provides at least one skin care benefit condition; and written instructions for administration to the subject in need.

Also provided by the present invention are methods for providing a benefit such as a skin care, skin supplement, hair care, oral care, wherein said methods comprise the topical application of a composition comprising an effective amount of an active ingredient selected from the group consisting of mevalonolactone, mevalonic acid, mevalonolactone monohydrate, and any combination thereof.

More specifically, the present invention discloses methods for providing a skin care benefit selected from the group consisting of skin moisturizing, skin exfoliation (also referred to as skin peeling, desquamation, skin shedding), skin resurfacing, skin regeneration, skin renewal, improving epidermal cell turnover, preventing or retarding the appearance of the signs of aging of the skin (retarding or reducing skin aging, anti-aging), reducing the appearance of skin wrinkles, skin rejuvenation, strengthening the skin barrier function, and any one combination thereof.

In one embodiment, the method is a method for providing at least one skin care benefit in a subject, comprising contacting a skin of said subject with a skin care composition comprising an active ingredient selected from the group consisting of mevalonolactone, mevalonic acid, mevalonolactone monohydrate, and any one combination thereof.

In one embodiment, the method is a method for improving the skin barrier function (providing an increased skin barrier function) of a skin in a subject, said method comprising contacting a skin of said subject with a skin care composition comprising an effective amount of an active ingredient selected from the group consisting of mevalonolactone, mevalonic acid, mevalonate, mevalonolactone monohydrate, or any one combination thereof, wherein the skin barrier function of the skin contacted with said composition is increased (strengthened) versus a control skin that was contacted with a placebo composition lacking said effective amount of said active ingredient.

In one embodiment, the method is a method for providing an increased skin exfoliation to a skin of a subject, said method comprising contacting a skin of said subject with a skin care comprising an effective amount of an active ingredient selected from the group consisting of mevalonolactone, mevalonic acid, mevalonate, mevalonolactone monohydrate, or any one combination thereof, wherein said effective amount of said active ingredient result in an increased skin exfoliation versus a control skin than was contacted with a placebo composition lacking said effective amount of said active ingredient.

In one embodiment, the method is a method for increasing the skin moisturization (water content of the skin) of a skin in a subject, said method comprising contacting a skin of said subject with a skin care composition comprising an effective amount of an active ingredient selected from the group consisting of mevalonolactone, mevalonic acid, mevalonate, mevalonolactone monohydrate, or any one combination thereof, and one or more dermatologically or cosmetically acceptable components, wherein the skin moisturization of the skin contacted with said composition is increased versus a skin than was contacted with a placebo composition lacking said effective amount of said active ingredient.

In one aspect, the present disclosure relates to skin aging caused by intrinsic and extrinsic factors, such as, but not limited to, oxidative damage, DNA damage, impaired DNA repair, impaired cell division, excessive inflammation, immune diseases, excessive cell death, sun, sunburn, collagen damage, elastin damage, senescence, telomere shortening, impaired expression of antioxidant enzymes, impaired activity of antioxidant enzymes, infrared radiation, heat, hormonal reasons, poor nutrition, temperature, tobacco smoking, stress, sleep deprivation, pollution, or alcohol consumption.

In one embodiment, the skin care benefit of a skin of a subject is attained by changes in the lipidic profile of the skin of said subject by administering to the skin an effective amount of mevalonolactone, mevalonic acid, mevalonate, salts of mevalonic acid, mevalonolactone monohydrate, and any one combination thereof.

In a further embodiment, the skin care benefit of a skin of a subject is attained by at least one change in lipid profile selected from the group consisting of an increase in hexosylceramide (HexCer), an increase in cholesterol esters (CE), an increase in triacylglycerol (TAG), an increase in phosphatidylcholines (PC), an increase in phosphatidate (PA), an increase in phosphatidylcholine-ether (PC-O), a decrease in diacylglycerol (DAG), a decrease in PG/CL, a decrease in phosphatidylethanolamine-ethers (LPE, LPE-O, PE-O), a decrease in phosphatidylinositol (PI), and any of combination thereof, by administering to the skin of said subject an effective amount mevalonolactone, mevalonic acid, mevalonate, salts of mevalonic acid, mevalonolactone monohydrate, and any one combination thereof.

In one embodiment, the skin care benefit of a skin of a subject is attained by changes in the lipidic profile of the skin of said subject by administering to the skin an effective amount of mevalonolactone.

In a further embodiment, the skin care benefit of a skin of a subject is attained by at least one change in lipid profile selected from the group consisting of an increase in hexosylceramide (HexCer), an increase in cholesterol esters (CE), an increase in triacylglycerol (TAG), an increase in phosphatidylcholines (PC), an increase in phosphatidate (PA), an increase in phosphatidylcholine-ether (PC-O), a decrease in diacylglycerol (DAG), a decrease in PG/CL, a decrease in phosphatidylethanolamine-ethers (LPE, LPE-O, PE-O), a decrease in phosphatidylinositol (PI), and any of combination thereof, by administering to the skin of said subject an effective amount mevalonolactone.

### Non-Limiting Examples of Compositions and Methods Disclosed Herein are as Follows:

1. A skin care composition for providing at least one skin care benefit in a subject, comprising an effective amount of an active ingredient selected from the group consisting, of mevalonolactone, mevalonic acid, mevalonate, salts of mevalonic acid, mevalonolactone monohydrate, and any one combination thereof, wherein said composition provides at least one skin care benefit to a skin of said subject.
1b. A skin care composition comprising an effective amount of an active ingredient selected from the group consisting of mevalonolactone, mevalonic acid, mevalonate, salts of mevalonic acid, mevalonolactone monohydrate, and any one combination thereof, for providing at least one skin care benefit in a subject.
1c. A skin care composition for providing at least one skin care benefit in a subject, comprising an effective amount of mevalonolactone, wherein said composition provides at least one skin care benefit to said subject.
1d. A skin care composition for providing at least one skin care benefit in a subject, comprising an effective amount of mevalonic acid, wherein said composition provides at least one skin care benefit to said subject.
1e. A skin care composition for providing at least one skin care benefit in a subject, comprising an effective amount of a salt of mevalonic acid, wherein said composition provides at least one skin care benefit to said subject.
1f. A skin care composition for providing at least one skin care benefit in a subject, comprising an effective amount of mevalonolactone monohydrate, wherein said composition provides at least one skin care benefit to said subject.
1g. A composition comprising an effective amount of an active ingredient selected from the group consisting of mevalonolactone, mevalonic acid, mevalonate, salts of mevalonic acid, mevalonolactone monohydrate, and any one combination thereof, for use in the treatment of a skin disorder of a subject, characterized in that the composition is for topical administration.
1h. A composition comprising an effective amount of an active ingredient selected from the group consisting of mevalonolactone, mevalonic acid, mevalonate, salts of mevalonic acid, mevalonolactone monohydrate, and any one combination thereof, for use in providing at least one skin benefit in a subject, characterized in that the composition is for topical administration.
1i. The composition of embodiment 1h, wherein the skin care benefit is selected from the group consisting of skin moisturizing, skin exfoliation (also referred to as skin peeling, desquamation, skin shedding), skin resurfacing, skin regeneration, skin renewal, improving epidermal cell turnover, preventing or retarding the appearance of the signs of aging of the skin (anti-aging), reducing the appearance of skin wrinkles, skin rejuvenation, strengthening the skin barrier function, or any one combination thereof.
1j. A skin care composition for providing at least one skin care benefit in a subject, comprising an effective amount of an active ingredient selected from the group consisting of mevalonolactone, mevalonic acid, mevalonate, salts of mevalonic acid, mevalonolactone monohydrate, and any one combination thereof, wherein said composition provides at least one skin care benefit to a skin of said subject, and wherein said active ingredient is produced by any one of the methods described herein.
2. The composition of any one of embodiments 1-1i, further comprising one or more dermatologically or cosmetically acceptable components.
2b. The composition of any one of embodiments 1-1i, further comprising at least one additional compound selected from the group consisting of a preservative, a pH adjuster, and one or more dermatologically or cosmetically acceptable component.
3. The composition of any one of embodiments 1-1i, wherein said effective amount of an active ingredient selected from the group consisting of mevalonolactone, mevalonic acid, mevalonate, salts of mevalonic acid, mevalonolactone monohydrate, and any one combination thereof, is between 0.01% to 10.0% on a weight basis relative to a total weight of said composition.
3b. The skin care composition of embodiment 1c, wherein said effective amount of mevalonolactone, is between 0.01% to 10.0% on a weight basis relative to a total weight of said composition.
3c. The skin care composition of embodiment 1d, wherein said effective amount of mevalonic acid, is between 0.01% to 10.0% on a weight basis relative to a total weight of said composition.
3d. The skin care composition of embodiment 1e, wherein said effective amount of a salt of mevalonic acid, is between 0.01% to 10.0% on a weight basis relative to a total weight of said composition.
3e. The skin care composition of embodiment 1f, wherein said effective amount of mevalonolactone monohydrate, is between 0.01% to 10.0% on a weight basis relative to a total weight of said composition.
3f. A skin care composition comprising between 0.01% to 10.0% mevalonolactone on a weight basis relative to a total weight of said composition.
3g. A skin care composition comprising between 0.01% to 10.0% mevalonic acid on a weight basis relative to a total weight of said composition.
4. The skin care composition of any one of embodiments 1-3g, wherein the at least one skin care benefit is selected from the group consisting of skin moisturizing, skin exfoliation (also referred to as skin peeling, desquamation, skin shedding), skin resurfacing, skin regeneration, skin renewal, improving epidermal cell turnover, preventing or retarding the appearance of the signs of aging of the skin (anti-aging), reducing the appearance of skin wrinkles, skin rejuvenation, strengthening the skin barrier function, or any one combination thereof.
5. The skin care composition of embodiment 4, wherein the at least one skin care benefit is an increased (strengthening the) skin barrier function.
6. The skin care composition of embodiment 4, wherein the at least one skin care benefit is skin moisturization (an increased skin water content).
7. The skin care composition of embodiment 4, wherein the at least one skin care benefit is skin exfoliation.
7b. A skin care composition for use in skin exfoliation treatment of a skin of a subject, comprising a dermatologically acceptable carrier and a concentration of at least 0.01%, 0.1%, 0.2%, 0.3%, 0.5%, 0.6%, 0.7%, 0.8%, 0.9%, 1% to at least 10% of mevalonolactone and/or mevalonic acid.
8. The skin care composition of embodiment 1 or embodiment 4, wherein the composition changes the lipidic profile of the skin (versus a control skin treated with the same composition lacking said mevalonolactone.).
8b. The skin care composition of embodiments 1 or 8, wherein said skin treated with said effective amount of mevalonolactone shows at least one change in lipid profile selected from the group consisting of an increase in hexosylceramide (HexCer), an increase in cholesterol esters (CE), an increase in triacylglycerol (TAG), an increase in phosphatidylcholines (PC), an increase in phosphatidate (PA), an increase in phosphatidylcholine-ether (PC-O), a decrease in diacylglycerol (DAG), a decrease in PG/CL, a decrease in phosphatidylethanolamine-ethers (LPE, LPE-O, PE-O), a decrease in phosphatidylinositol (PI), and any of combination thereof, versus a control skin treated with the same composition lacking said mevalonolactone.
8c. The skin care composition of embodiment 1 or embodiment 4, wherein the composition maintains or increases the skin mechanical and biological properties leading to an improved healthy look, also called anti-aging effect.
8d. The skin care composition of any one of embodiment 1-8b, wherein said composition is a clear aqueous solution.
8e. The skin care composition of any one of the preceding embodiments, wherein the mevalonolactone is prepared from a mevalonolactone monohydrate source or a mevalonic acid source.
9. The skin care composition of any one of embodiments 1-8d, wherein the composition is selected from the group consisting of an aqueous solution, an emulsion, a serum, a jelly, a mask, a patch, a face mask, a peel-off mask, a lotion, a topical moisturizer, a cream, a paste, a balm, an ointment, a pomade, a gel, a liquid, a spray, a foam, a kits, a sun care, a baby care, a hair care, or any one combination thereof.
10. A method for providing at least one skin care benefit in a subject, comprising contacting a skin of said subject with a skin care composition comprising an active ingredient selected from the group consisting of mevalonolactone, mevalonic acid, mevalonolactone monohydrate, and any one combination thereof.
10b. A method for providing at least one skin care benefit in a subject, comprising administering to a skin of said subject a skin care composition comprising an effective amount of an active ingredient selected from the group consisting of mevalonolactone, mevalonic acid, mevalonolactone monohydrate, and any one combination thereof.
10c. A method for providing at least one skin care benefit in a subject, comprising contacting a skin of said subject with one or more dermatologically or cosmetically acceptable components and an active ingredient selected from the group consisting of mevalonolactone, mevalonic acid, mevalonolactone monohydrate, and any one combination thereof.
10d. Use of an effective amount of an active ingredient selected from the group consisting of mevalonolactone, mevalonic acid, mevalonate, mevalonolactone monohydrate, and any one combination thereof, in a skin care, wherein said composition provides at least one skin care benefit
10e. The use of embodiment 10d, wherein the at least one skin care benefit is selected from the group consisting of skin moisturizing, skin exfoliation (also referred to as skin peeling, desquamation, skin shedding), skin resurfacing, skin regeneration, skin renewal, improving epidermal cell turnover, preventing or retarding the appearance of the signs of aging of the skin, reducing the appearance of skin wrinkles, skin rejuvenation, strengthening the skin barrier function, or any one combination thereof.
11. The method of embodiment 10, wherein the at least one skin care benefit is selected from the group consisting of skin moisturizing, skin exfoliation (also referred to as skin peeling, desquamation, skin shedding), skin resurfacing, skin regeneration, skin renewal, improving epidermal cell turnover, preventing or retarding the appearance of the signs of aging of the skin (anti-aging), reducing the appearance of skin wrinkles, skin rejuvenation, strengthening the skin barrier function, and any one combination thereof.
12. A method for improving (increasing) the skin barrier function (providing an improved (increased) skin barrier function) of a skin in a subject, said method comprising contacting a skin of said subject with a skin care composition comprising an effective amount of an active ingredient selected from the group consisting of mevalonolactone, mevalonic acid, mevalonate, mevalonolactone monohydrate, or any one combination thereof.
12a. A method for improving (increasing) the skin barrier function (providing an improved (increased) skin barrier function) of a skin in a subject, said method comprising contacting a skin of said subject with a skin care composition comprising an effective amount of mevalonolactone.
12b. A method for improving (increasing) the skin barrier function (providing an improved (increased) skin barrier function) of a skin in a subject, said method comprising contacting a skin of said subject with a skin care composition comprising an effective amount of mevalonic acid.
12c. The method of embodiment 12, wherein the skin barrier function of the skin contacted with said composition is increased (strengthened) versus a control skin that was contacted with a placebo composition lacking said effective amount of said active ingredient.
12d. The method of embodiment 12, wherein the skin care composition further comprises one or more dermatologically or cosmetically acceptable components.
13. A method for providing an increased skin exfoliation to a skin of a subject, said method comprising contacting a skin of said subject with a skin care comprising an effective amount of an active ingredient selected from the group consisting of mevalonolactone, mevalonic acid, mevalonate, mevalonolactone monohydrate, or any one combination thereof, wherein said effective amount of said active ingredient result in an increased skin exfoliation versus a control skin than was contacted with a placebo composition lacking said effective amount of said active ingredient.
13b. A method for providing an improved skin desquamation to a subject said method comprising contacting a skin of said subject with a skin care comprising an effective amount of an active ingredient selected from the group consisting of mevalonolactone, mevalonic acid, mevalonate, mevalonolactone monohydrate, or any one combination thereof, wherein said effective amount of said active ingredient result in an increased skin desquamation versus a control skin than was contacted with a placebo composition lacking said effective amount of said active ingredient.
14. A method for increasing the skin moisturization (water content of the skin) of a skin in a subject, said method comprising contacting a skin of said subject with a skin care composition comprising an effective amount of an active ingredient selected from the group consisting of mevalonolactone, mevalonic acid, mevalonate, mevalonolactone monohydrate. or any one combination thereof, and one or more dermatologically or cosmetically acceptable components. wherein the skin moisturization of the skin contacted with said composition is increased versus a skin than was contacted with a placebo composition lacking said effective amount of said active ingredient.
15. The method of anyone of embodiments 10-13, wherein said skin care composition further comprises one or more dermatologically or cosmetically acceptable component.
16. The method of anyone of embodiments 10-13, wherein the effective amount of the active ingredient is at least 0.01%, 0.02%, 0.03%, 0.04%, 0.05%, 0.06%, 0.07%, 0.08%, 0.09%, 0.1%, 0.2%, 0.3%, 0.5%. 0.6%, 0.7%, 0.8%, 0.9%, 1%, 2%, 3%, 4%, 5%, 6%, 7%, 8%, 9% up to 10% on a weight basis relative to a total weight of said composition.
17. A pharmaceutical or home care or nutritional or personal care or cosmetic composition comprising mevalonolactone (MVL) produced from any one of the methods described hereon.
18. A pharmaceutical or home care or nutritional or personal care or cosmetic composition comprising mevalonolactone (MVL) produced from a method for producing a water solubilized mevalonolactone from an aqueous solution wherein the method comprises: a) producing a crystalline form of a salt of mevalonic acid (X-MVA) from an aqueous solution by subjecting the aqueous solution comprising said salt of mevalonic acid to a nanofiltration to produce a permeate and crystallizing said salt of mevalonic acid from said permeate by water solvent crystallization to produce crystals of said salt of mevalonic acid; and, b) dissolving the crystals of (a) in water to produce a water solubilized salt of mevalonic acid and subjecting said liquid to cation exchange thereby converting said water solubilized salt of mevalonic acid to water solubilized mevalonolactone.
19. A pharmaceutical or home care or nutritional or personal care or cosmetic composition comprising mevalonolactone (MVL) produced from a method for producing mevalonolactone from an aqueous solution comprising a salt of mevalonic acid salt (X-MVA), comprising subjecting the aqueous solution comprising said salt of mevalonate to cation exchange thereby converting said aqueous solution comprising a salt of mevalonate to an aqueous solution comprising mevalonolactone (MVL).

### EXAMPLES

In the following Examples, unless otherwise stated, parts and percentages are by weight and degrees are Celsius. It should be understood that these Examples, while indicating embodiments of the disclosure, are given by way of illustration only. From the above discussion and these Examples, one skilled in the art can make various changes and modifications of the disclosure to adapt it to various usages and conditions. Such modifications are also intended to fall within the scope of the appended claims.

### Example 1

### Sodium Mevalonate (Na-MVA) Liquid Nanofiltration (NF) with Tight Nanofiltration Membranes

This example describes the nanofiltration of an aqueous solution comprising an organic acid or salt thereof, such as sodium mevalonate (Na-MVA) liquid nanofiltration using a tight nanofiltration membrane.

The process equipment included a plate and frame filtration unit (Alfa Laval Labstak M20), feed and diafiltration pump, heat exchanger, cooling unit, 70-liter feed tank as well as inlet and outlet pressure gauges and pressure control valve. The total membrane area was 0.65 m². The membranes installed were, Vesal-5 DL (Suez, approximate molecular weight cut-off of 150-300 Dalton, >98% MgSO₄ retention at 25° C.) and XN45 (TriSep®/Microdyn Nadir, approximate molecular weight cut-off of 300-500 Dalton, 90-96% MgSO₄ retention at 25° C). MgSO₄ retention specified at 1-2 g/l concentration in 7-8 bar, 25° C., pH 6-8 and 10-25% recovery.

Ultrafiltration permeate from Escherichia Coli based fermentation of sodium mevalonate was used as a feed. The aim was to pass the sodium mevalonate while retaining impurities (other salts. color. antifoam, protein).

63.4 kg of feed was fed into a 70-liter feed tank. The dry feed concentration was 6.8 g/100 g based on sucrose RDS, conductivity was 17.7 mS/cm and the pH was 7.8. Permeate was collected from DL membranes and permeate from XN45 membranes was recycled back to the feed tank. The feed was composed as set shown in Table 1, wherein the HPLC and IC analyses are given on %/sucrose RDS basis.

TABLE 1
Feed composition (%)

| | |
|---|---|
| Sodium mevalonate, % | 66.4 |
| Glucose, %* | 0.6 |
| Acetate, %** | 2.1 |
| Phosphate, %** | 1.6 |
| Sulphate, %** | 0.5 |
| Others, % | 28.8 |

*HPLC (Rezex ROA-Organic Add H+ (8%) column)
**Ion chromatography

The feed was kept at 50-68° C. and water was used for diafiltration. The filtration pressure was 20-33 Bar and concentrate DS (dry substance) concentration controlled to keep flux above 7 kg/m²/h (the minimum flux point observed).

After batch filtration, three permeate fractions and a final concentrate fraction were collected. The result including HPLC and IC analyses on %/sucrose RDS basis for the permeate fraction and final concentrate are shown in Table 2.

TABLE 2
HPLC analysis

| | | | | Final |
|---|---|---|---|---|
| | Total perme- | Total perme- | Total perme- | concen- |
| | ate 1 | ate 2 | ate 3 | trate |
| mass, kg | 31.0 | 31.6 | 4.5 | 17.1 |
| Dry solids, g/100g | 2.0 | 3.6 | 3.4 | 13.9 |
| Conductivity mS/cm | 8.1 | 11.3 | 10.9 | 26.2 |
| Sodium mevalonate, | 52.1 | 86.9 | 86.2 | 63.3 |
| %* | | | | |
| Glucose, %* | 1.2 | 2.0 | 0.9 | 0.4 |
| Acetate, %** | 7.6 | 2.7 | 0.7 | 0.3 |
| Phosphate, %** | 0.5 | 0.8 | 1.1 | 2.0 |
| Sulphate, %** | 0.1 | 0.1 | 0.1 | 0.8 |
| Others, % | 38.6 | 7.5 | 11.0 | 33.2 |

*HPLC (Rezex ROA-Organic Acid H+ (8%) column).
**Ion chromatography

The overall mevalonate yield calculated was 49.9%. Average permeate sodium mevalonate purity was 75.6%.

Sulfate removal was 78.1% and phosphate removal was 71.0% calculated from feed and concentrate samples. Sodium mevalonate retention was measured from 5 points for DL membranes and average values was 76.7%. DL and XN45 mevalonate retentions were measured in parallel from the 4^{th} sample point after 41 kg permeate was collected and retentions were 71.0% and 41.8% for DL and XN45 respectively.

### Example 2

### Sodium Mevalonate Nanofiltration with Open Nanofiltration (NF) Membranes

This example describes the nanofiltration an aqueous solution comprising an organic acid or salt thereof, such as sodium mevalonate liquid nanofiltration using open nanofiltration membranes.

The process equipment included a plate and frame filtration unit (Alfa. Laval Labstak M20), feed and diafiltration pump, heat exchanger, cooling unit, 100-liter feed tank as well as inlet and outlet pressure gauges and pressure control valve. The total membrane area was 0.72 m². The membranes installed were, XN45 (TriSep®/Microdyn Nadir, approximate molecular weight cut-off of 300-500 Dalton, 90-96% MgSO₄ retention at 25° C.). MgSO₄ retention specified at 1-2 g/l concentration in 7-8 bar, 25° C., pH 6-8 and 10-25% recovery.

Ultrafiltration permeate from Escherichia Coli based fermentation of sodium mevalonate was used as a feed. The aim was to pass the sodium mevalonate while retaining impurities (other salts, color, antifoam, protein).

80.0 kg of feed was fed into a 100-liter feed tank. The feed concentration was 6.6 g/100 g based on sucrose RDS. pH was adjusted with NaOH to 8.8. Antifoam Foamblast 882 concentration was measured to be 1337 mg/L. The feed was composed as shown in Table 3, wherein the HPLC and IC analyses are given on %/sucrose RDS basis.

TABLE 3
Feed composition (%)

| | |
|---|---|
| Sodium mevalonate, %* | 55.6 |
| Glucose, %* | 0.8 |
| Acetate, %** | 2.2 |
| Phosphate, %** | 1.1 |
| Sulphate, %** | 0.5 |
| Others, % | 39.8 |

*HPLC (Rezex ROA-OrganicAcid H+ (8%) column).
**Ion chromatography

The feed was kept at 46-51° C. The filtration pressure was 0-25 Bar used to control flux above 7 kg/m²/h (the minimum flux point observed).

After batch filtration, two permeate fractions and final concentrate fraction were collected. The result including HPLC and IC analyses on %/sucrose RDS basis for the permeate fraction and final concentrate are shown in Table 4.

TABLE 4
HPLC analysis

| | Total permeate 1 | Total permeate 2 | Final concentrate |
|---|---|---|---|
| mass, kg | 67.3 | 9.5 | 5.3 |
| Dry solids, g/100 g | 4.1 | 8.6 | 16.3 |
| Sodium | 65.3 | 69.3 | 35.1 |
| mevalonate, %* | | | |
| Glucose, %* | 1.0 | 0.7 | 0.6 |
| Acetate, %** | 3.4 | 1.3 | 0.3 |
| Phosphate, %** | 0.4 | 0.6 | 2.3 |
| Sulphate, %** | 0.2 | 0.1 | 0.9 |
| Others, % | 29.7 | 28.0 | 60.8 |

*HPLC (Rezex ROA-Organic Acid H+ (8%) column).
**Ion chromatography

The overall mevalonate yield calculated was 82.1%. Antifoam concentration measured from evaporated permeate sample in 29.5% concentration was 84.4 mg/l meaning 99% antifoam removal from product permeate fraction. Final concentrate antifoam concentration measured was 13230.0 mg/l. Sodium mevalonate retention was measured from 3 points and average value was 23.6%.

### Example 3

### Sodium Mevalonate Liquid Nanofiltration with Open Nanofiltration (NF) Membranes

This example describes the nanofiltration an aqueous solution comprising an organic acid or salt, thereof, such as sodium mevalonate liquid nanofiltration using open nanofiltration membranes.

The process equipment included a plate and frame filtration unit (Alfa Laval Labstak M20), feed, and diafiltration pump, heat exchanger, cooling unit, 100-liter feed tank as well as inlet and outlet pressure gauges and pressure control valve. The total membrane area was 0.72 m². The membranes installed were, XN45 (TriSep®/Microdyn Nadir, approximate molecular weight cut-off of 300-500 Dalton, 90-96% MgSO₄ retention at 25° C.). MgSO₄ retention specified at 1-2 g/l concentration in 7-8 bar, 25° C., pH 6-8 and 10-25% recovery.

Ultrafiltration permeate from Escherichia Coli based fermentation of sodium mevalonate was used as a feed. The aim was to pass the sodium mevalonate while retaining impurities (other salts, color, antifoam, protein).

79.7 kg of feed was fed into a 100-liter feed tank. The feed concentration was 7.0 g/100 g. pH was adjusted with 50 grams of 30% NaOH to 8.25. The feed was composed as shown in Table 5, wherein the HPLC analyses are given on %/sucrose RDS basis (Table 6).

TABLE 5
Feed composition (%)

| | |
|---|---|
| Sodium mevalonate, %* | 65.1 |
| Glucose, %* | 0.0 |
| Acetate, %** | 1.8 |
| Phosphate, %** | 0.3 |
| Sulphate, %** | 0.5 |
| Others, % | 32.3 |

*HPLC (Rezex ROA-Organic Acid H+ (8%) column)
**Ion chromatography

The feed was kept at 46-51° C. The filtration pressure was 10-25 Bar used to control flux above 7 kg/m²/h (the minimum flux point observed).

After batch filtration, permeate fraction and final concentrate fraction were collected. Part of total permeate was evaporated to 28.7% dry solids. The result including HPLC analyses on %/sucrose RDS basis for the permeate fraction and final concentrate are shown in Table 6.

TABLE 6
HPLC analysis

| | Total | Final | Evaporated |
|---|---|---|---|
| | permeate | concentrate | permeate |
| mass, kg | 75.0 | 4.7 | — |
| Dry solids. g/100 g | 6.1 | 17.6 | 30.2 |
| Sodium mevalonate, | 69.3 | 47.6 | 75.1 |
| %* | | | |
| Acetate, %** | 2.1 | 0.4 | 2.5 |
| Phosphate, %** | 0.1 | 0.5 | 0.2 |
| Sulphate, %“ | 0.3 | 0.9 | 0.4 |
| Others, % | 28.3 | 50.5 | 21.8 |

*HPLC (Rezex ROA-Organic Acid H+ (8%) column),
**IC

The overall mevalonate yield calculated was 86.8%.

### Example 4

### Sodium Mevalonate Nanofiltration with Open Spiral Wound Nanofiltration (NF) Membranes

This example describes the nanofiltration of an aqueous solution comprising an organic acid or salt thereof, such as sodium mevalonate liquid nanofiltration using open spiral wound nanofiltration membranes.

The process equipment included a plate and frame filtration unit (Alfa Laval Labstak M20), feed and diafiltration pump, heat exchanger, cooling unit, 1000-liter feed tank as well as inlet and outlet pressure gauges and pressure control valve. The total membrane area was 14.8 m². The membranes installed were, 4040 XN45 with 31 mil spacer (TriSep®/Microdyn Nadir, approximate molecular weight cut-off of 300-500 Dalton, 90-96% MgSO₄ retention at 25° C.). MgSO₄ retention specified at 1-2 g/l concentration in 7-8 bar, 25° C., pH 6-8 and 10-25% recovery.

Ultrafiltration permeate from Escherichia Coll based fermentation of sodium mevalonate was used as a feed. The aim was to pass the sodium mevalonate while retaining impurities (other salts, color, antifoam, protein).

600 kg of feed was fed into a 1000-liter feed tank. The feed concentration was 6.7 g/100g. pH was adjusted with 30% NaOH to 8.5. The feed was composed as shown in Table 7, wherein the HPLC analyses are given on %/sucrose RDS basis.

TABLE 7
Feed composition (%)

| | |
|---|---|
| Sodium mevalonate, % | 64.5 |
| Acetate, % | 1.2 |
| Others, % | 34.3 |

The feed was kept in 45-52° C. The filtration pressure was 10-25 Bar used to control flux above 5 kg/m²/h (the minimum flux point observed).

After batch filtration, permeate fraction and final concentrate fraction were collected. Total permeate was evaporated to 39.1% dry solids. The result including HPLC analyses on %/sucrose RDS basis for the permeate fraction and final concentrate are shown in Table 8.

TABLE 8
HPLC analysis

| | Total | Final | Evaporated |
|---|---|---|---|
| | permeate | concentrate | permeate |
| mass, kg | 606.0 | 45 | — |
| Dry solids, g/100 g | 5.5 | 16.7 | 39.1 |
| Sodium mevalonate, % | 68.8 | 52.2 | 75.5 |
| Acetate, % | 1.8 | 0.7 | 1.9 |
| Others, % | 29.4 | 47.1 | 22.6 |

The overall mevalonate yield calculated was 87.5%.

### Example 5

### Water Solvent Crystallization of Sodium Mevalonate (Na-MVA)

This example describes the water solvent crystallization of organic acids or salts thereof, such as water solvent crystallization of sodium mevalonate (Na-MVA), and preparation of sodium mevalonate seed crystals.

The crystallization feed material was an aqueous syrup comprising sodium mevalonate. The sodium mevalonate purity of the syrup was 96% on DS. An aqueous syrup comprising sodium mevalonate ≥95% on DS can be produced by dissolving known, commercially available, sodium mevalonate salt in deionized water or by mixing known, commercially available, sodium mevalonate salt with nanofiltration permeate, comprising sodium mevalonate, prepared in accordance with Examples 1-4.

The feed syrup was evaporated (Rotavapor R-151) at a temperature of 70° C. When the DS (dry substance) was about 54%, crystals formed by spontaneous nucleation. Evaporation was continued after nucleation at 70° C. for 2 hr. until the DS of the crystal mass was 81.8%.

The evaporated crystal mass was moved to a 2-liter cooling crystallizer. The crystal mass was kept at 70° C. for 1 hr., and then cooled to a temperature of 40° C. within 20 hr. under continuous stirring.

930 g of the resulting crystal mass was centrifuged with 20 mL wash water (batch-wise centrifuge, basket diameter 22.5 cm, 3500 rpm, 5 min). The mevalonate yield in centrifugation was 45%, the sodium mevalonate purity of the centrifugation cake was ≥98% on DS, and the sodium mevalonate purity of the centrifugation mother liquor was 94% on DS.

The crystal cake was dried in a heating chamber at 60° C. for 19 hr. The water content of the dried cake was ≤0.2 wt. %.

Sodium mevalonate crystals prepared in accordance with this process were used as seed crystals in Examples 6, 7, and 8. Before being used, the crystals were grinded in a porcelain mill.

### Example 6

### Water Solvent Crystallization of Sodium Mevalonate (Na-MVA)

This example describes the water solvent crystallization of organic acids or salts thereof, such as water solvent crystallization of sodium mevalonate (Na-MVA).

The crystallization feed material was evaporated nanofiltration permeate, prepared in accordance with Example 4. Before crystallization, the syrup was treated with active carbon powder to remove color. The carbon treatment was carried out by dosing 600 g of Norit DX 1 carbon powder to 76 kg of evaporated syrup (about 20 g of carbon powder per 1 kg of DS). The syrup was heated to a temperature of 50° C. and kept stirring at constant temperature for 45 min. The carbon powder was then separated from the syrup with filter aid filtration (Seitz depth filter, 1.1 kg of Kenite 300 filter aid). The sodium mevalonate purity of the resulting syrup was 73% on DS, the color was 6300 ICUMSA, and the DS was 24.1%.

41 kg of the carbon treated feed syrup was evaporated to a DS of 86.0% (Luwa thin film evaporator NL3-210/1600/10 and Rotavapor R-153 evaporator). 4 g of 10% Struktol J 650 antifoam was added during evaporation to avoid foaming. 11.6 kg of the resulting syrup was moved to a 10-liter cooling crystallizer and seeded two times at a temperature of 70° C.: at first, with 0.3 g of sodium mevalonate dry seed (prepared in accordance with Example 5), and then with 2.0 g of sodium mevalonate dry seed after 0.6 hr from the first seeding. The first seeding resulted in very little crystal formation.

The seeded syrup was cooled to a temperature of 48° C. within 16 hr under continuous stirring. After cooling to 48° C., the crystal mass was kept at 48° C. for 2 hr.

10.7 kg of the resulting crystal mass was centrifuged with 170 mL wash water (batch-wise centrifuge, basket diameter 40.5 cm, 2050 rpm, 10 min). The mevalonate yield in centrifugation was 33%. The sodium mevalonate purity of the centrifugation cake was 94% on DS, the water content of the non-dried cake was 5.3 wt. %, and the color was 5200 ICUMSA. The sodium mevalonate purity of the centrifugation mother liquor was 68% on DS and the color was 82000 ICUMSA.

### Example 7

### Water Solvent Crystallization of Sodium Mevalonate (Na-MVA)

This example describes the water solvent crystallization of organic acids or salts thereof, such as water solvent crystallization of sodium mevalonate (MVA).

The crystallization feed material was evaporated nanofiltration permeate, prepared in accordance with Example 4, which was treated with active carbon in accordance with the procedure described in Example 6 (20 g of Norit DX 1 carbon powder per 1 kg of DS, contact time 45 min, contact temperature 50° C., carbon powder separated by using Seitz depth filter with Kenite 300 filter aid). The sodium mevalonate purity of the feed was 73% on DS and the color was 6300 ICUMSA.

The feed syrup was evaporated to a DS of 86.2% (Luwa thin film evaporator NL3-210/1600/10 evaporator and Rotavapor R-153 evaporator). 11.7 kg of the resulting syrup was moved to a 10-liter cooling crystallizer and seeded with 2.0 g of sodium mevalonate dry seed (prepared in accordance with Example 5) at a temperature of 69° C. The seeded syrup was cooled to a temperature of 44° C. within 16 hr under continuous stirring. The crystal mass was then diluted to a DS of 85.4% by adding deionized water. The diluted crystal mass was cooled to a temperature of 40° C. within 3 hr and kept at 40° C. for 2 hr.

10.9 kg of the resulting crystal mass was centrifuged with 170 mL wash water (batch-wise centrifuge, basket diameter 40.5 cm, 2050 rpm, 10 min). The mevalonate yield in centrifugation was 37%. The sodium mevalonate purity of the centrifugation cake was 95% on DS, the water content of the non-dried cake was 7.0 wt. %, and the color was 1100 ICUMSA. The sodium mevalonate purity of the centrifugation mother liquor was 67% on DS and the color was 14000 ICUMSA.

### Example 8

### Water Solvent Crystallization of Sodium Mevalonate (Na-MVA)

This example describes the water solvent crystallization of organic acids or salts thereof, such as water solvent crystallization of sodium mevalonate (Na-MVA), from a syrup prepared by dissolving crystal cake in deionized water.

Sodium mevalonate crystals from Example 6 and Example 7 were combined and dissolved in deionized water. The resulting solution was treated with active carbon in accordance with the procedure described in Example 6 (20 g of Norit DX 1 carbon powder per 1 kg of DS, contact time 45 min, contact temperature 50° C., carbon powder separated by using Buchner filtration with Kenite 300 filter aid), filtered through a 0.2 μm sterile filter, and evaporated to a DS of 83.5% (Rotavapor R-151 evaporator). The sodium mevalonate purity of the syrup was 96% on DS and the color was 1300 ICUMSA.

5.6 kg of the evaporated syrup was moved to a 6-liter cooling crystallizer and seeded with 1.6 g of sodium mevalonate dry seed (prepared in accordance with Example 5) at a temperature of 68° C. The seeded syrup was cooled to a temperature of 40° C. within 18 hr under continuous stirring. After cooling to 40° C., the crystal mass was kept at 40° C. for 4 hr.

5.1 kg of the resulting crystal mass was centrifuged with 150 mL wash water (batch-wise centrifuge, basket diameter 40.5 cm, 2050 rpm, 10 min). The mevalonate yield in centrifugation was 42%. The sodium mevalonate purity of the centrifugation cake was ≥198% on DS, the water content of the non-dried cake was 1.5 wt. %, and the color was 100 ICUMSA. The sodium mevalonate purity of the centrifugation mother liquor was 94% on DS and the color was 3400 ICUMSA.

### Example 9

### Cation Exchange of Crystallized Na-MVA to Mevalonolactone (MVL)

This example describes the cation exchange of a liquid solution of dissolved Na-MVA crystals to produce a dilute liquid comprising mevalonate (MVL), which can optionally be concentrated by evaporation.

The process equipment included a jacketed glass column with 45 mm inner diameter and 1000 mm total length, a feed vessel placed above the column, a peristaltic pump on the output of the column and a Büchi rotavapor 8200 rotary lab evaporator. 1 liter of Dowex 88 Strong acid cation ion exchange resin was loaded into the column to give about 700 mm bed length. Resin was backwashed and regenerated to H⁺-form by using 4 liters (4 Bed volumes) of 5% H₂SO₄ at a flow rate of 2 BV/h in 25° C. temperature.

Sodium mevalonate crystals from a water based crystallization process with about 98%IDS purity were dissolved in water to form a 24.2% solution. The aim was to exchange the sodium cation and produce a pure mevalonic acid or mevalonolactone syrup.

A feed solution of 957 grams was passed through the column at room temperature (about 25° C.) at a rate of 1 BV/h and elution continued using water. pH of feed was 9.78 and conductivity 39.4 mS/cm. Product collection was started when column output brix was 0.4% and ended when output brix was 1.2%. 1322 grams of product was collected with slight coloring. The columns were again regenerated to H⁺-form and material was passed through to column in room temperature (about 25° C.) at a rate of 1 BV/h. Product collection was started when column output brix was 3.0% and ended when output brix was 1.4%. 1641 grams of product with no color was collected. The material was evaporated in a rotary evaporator to 99.1% KF-DS with 95.5%/DS MVL purity.

The results including HPLC analyses on %/dry substance basis for the feed and ion exchange (IEX) products are shown in Table 9.

TABLE 9
HPLC analysis

| | Feed | IEX product 1 | IEX product 2 |
|---|---|---|---|
| mass, g | 957.4 | 1322.0 | 1641.0 |
| Dry solids, g/100 g (KF-DS) | 24.2 | 13.7 | 10.4 |
| Conductivity mS/cm | 39.4 | 2.5 | 4.5 |
| pH | 9.8 | 2.9 | 1.8 |
| Mevalonolactone, % | 75.0 | 94.3 | 97.6 |
| Others in HPLC, % | 1.5 | 2.3 | 2.3 |

The overall mevalonolactone yield calculated for the IEX process was 95.6%.

The specific optical rotation of a syrup comprising mevalonolactone produced in accordance with this process was −34.7° (water, c=1, 20° C.).

### Example 10

### Cation Exchange of Na-MVA Solutions to Mevalonolactone (MVL)

This example describes the cation exchange of an aqueous solution containing Na-MVA to produce an aqueous comprising mevalonate (MVL)), which can optionally be concentrated by evaporation.

The process equipment included 100 liter tank, Seitz depth filter, three jacketed glass columns with 130 mm inner diameter and 1500 mm total length, a feed vessel placed above the column, a peristaltic pump on the output of the column and a LUWA wiped film evaporator. 13 liters of Dowex 88 Strong acid cation ion exchange resin (Dow) was loaded into each of the columns to give about 1000 mm bed length. Resins were backwashed and regenerated in series to H⁺-form by using 130 liters (about 3.3 Bed volumes) of 5% H₂SO₄ at a flow rate of 2 BV/h in 25° C. temperature.

The aim was to exchange the sodium cation and produce a mevalonic acid or mevalonolactone syrup for further purification. Powdered active carbon was used to remove color on possible precipitation from the IEX feed.

Nanofiltration permeate having 75.5%/DS Na-MVA purity and 10986 ICUMSA unit color was evaporated to 39.1% brix. 76.2 kg of the solution was heated to 50-60° C. temperature and 2%/DS of Norit DX1 powdered active carbon was added and mixed for 45 min. The suspension was filtered using Seitz depth filter with T2600 filtration sheets (Pall) with 0.56 m² filtration area and 1 kg/m² of Kenite 300 (IMERYS Filtration) diatomaceous earth filter aid as pre-coat. 110 liters of Filtrate with 24.1% brix was evaporated to 53% brix solution with 6496 ICUMSA unit color. 14.8 kg of this Material was diluted to 39.8% brix for IEX feed and heated to 50° C.

A feed solution of 20.05 kg was passed through to columns at 50° C. at a rate of 2 BV/h and elution continued using water. Product collection was started when last column output brix was 0.5% and ended when output brix was 4.0% and pH 2.5. Product was collected to two fractions. Fractions were combined and evaporated. Evaporated combined IEX product had color of 2362 ICUMSA units and MVL purity of about 76%/DS

The results of the feed and IEX products are set forth in Table 10.

TABLE 10
Feed and IEX products

| | Feed | IEX product 1 | IEX product 2 |
|---|---|---|---|
| mass, kg | 20.1 | 18.1 | 17.9 |
| Dry solids, (KF-DS) | 39.8 | 12.0 | 22.7 |
| Conductivity mS/cm | 32.3 | 4.1 | 3.9 |
| pH | 8.5 | 1.9 | 1.8 |

### Example 11

### Cation Exchange of Na-MVA Solutions to Mevalonolactone (MVL)

This example describes the cation exchange of an aqueous solution containing Na-MVA to produce an aqueous comprising mevalonate (MVL), which can optionally be concentrated by evaporation.

The process equipment included 100 liter tank, Seitz depth filter, three jacketed glass columns with 130 mm inner diameter and 1500 mm total length, a feed vessel placed above the column, a peristaltic pump on the output of the column and a LUWA wiped film evaporator. 13 liters of Dowex 88 Strong acid cation ion exchange resin (Dow) was loaded into each of the columns to give about 1000 mm bed length. Resins were backwashed and regenerated in series to H⁺-form by using 130 liters (about 3.3 Bed volumes) of 5% H₂SO₄ at a flow rate of 2 BV/h in 25° C. temperature.

The aim was to exchange the sodium cation and produce a mevalonic acid or mevalonolactone syrup for further purification. Powdered active carbon was used to remove color on possible precipitation from the !EX feed.

Nanofiltration permeate having 78.8%/DS Na-MVA, purity and 8887 ICUMSA unit color was evaporated to 42.0% brix. 41.4 kg of the solution was heated to 55-58° C. temperature and 2.2%/DS of Norit DX1 powdered active carbon was added and mixed for 70 min. The suspension was filtered using Seitz depth filter with T2600 filtration sheets (Pall) with 0.28 m² filtration area and 1 kg/m² of Kenite 300 (IMERYS Filtration) diatomaceous earth filter aid as pre-coat. 56 liters of Filtrate with 23.8% brix and 2124 ICUMSA unit color. Material was evaporated to 31.1% brix for IEX feed.

A feed solution of 35.7 kg was passed through to columns at 25° C. at a rate of 2 BV/h and elution continued using water. Product collection was started when last column output brix was 0.5% and ended when output brix was 2.0% and pH reached 2.5. Product and residual fractions were collected. Evaporated IEX product had MVL purity of about 78-83%/DS. The results the feed and IEX products are set forth in the Table 11.

TABLE 11
Feed and IEX products

| | Feed | IEX product | Residual Fraction |
|---|---|---|---|
| mass, kg | 35.7 | 34.1 | 14.3 |
| Dry solids, (KF-DS) | 31.1 | 18.0 | 16.4 |
| Conductivity mS/cm | 42.8 | 6.9 | 6.8 |
| pH | 7.6 | 1.7 | 3.6 |

### Example 12

### Cation Exchange of Na-MVA Solutions to Mevalonolactone (MVL)

This example describes the cation exchange of an aqueous solution containing Na-MVA to produce an aqueous comprising mevalonate (MVL), which can optionally be concentrated by evaporation.

The process equipment included three jacketed glass columns with 130 mm inner diameter and 1500 mm total length, a feed vessel placed above the column, a peristaltic pump on the output of the column. 13 liters of Dowex 88 Strong acid cation ion exchange resin (Dow) was loaded into each of the columns to give about 1000 mm, bed length. Resins were backwashed and regenerated in series to H⁺-form by using 130 liters (about 3.3 Bed volumes) of 5% H₂SO₄ at a flow rate of 2 BV/h in 25° C. temperature.

The aim was to exchange the sodium cation and produce a mevalonic acid or mevalonolactone symp for further purification.

Na-MVA crystallization end mother liquor with 68.2%!DS Na-MVA purity and 82952 ICUMSA unit color was diluted to 39.4% brix.

A feed solution of 17.0 kg was passed through to column in 25° C. at a rate of 2 BV/h and elution continued using water. Product collection was started when last column output brix was 0.8% and ended when output brix was 2.0%. Product was collected to three fractions. Fractions were combined and evaporated. Evaporated combined IEX product had MVL purity of about 70-73%/DS The results the feed and IEX products are set forth in Table 12.

TABLE 12
Feed and IEX products

| | | IEX | IEX | IEX |
|---|---|---|---|---|
| | Feed | product 1 | product 2 | product 3 |
| mass, kg | 17.0 | 18.5 | 10.6 | 6.9 |
| Dry solids, (KF-DS) | 39.4 | 16.7 | 17.4 | 5.1 |
| Conductivity mS/cm | 33.1 | 5.9 | 4.3 | 1.3 |
| pH | 7.3 | 1.7 | 1.8 | 2.4 |

### Example 13

### Water Solvent Crystallization of Mevalonolactone Monohydrate and Preparation of MVL*H₂O Seed Crystals

This example describes the water solvent crystallization of mevalonolactone monohydrate and preparation of mevalonolactone monohydrate seed crystals.

A high purity syrup of MVL was prepared by methods as described herein (Examples 1-9). Unexpectedly and surprisingly, it was observed that when this high MVL purity syrup was cold stored (at about 6° C.) for several weeks, mevalonolactone monohydrate (MVL*H₂O) crystals formed spontaneously by itself.

The MVL purity of the syrup, 98% of DS (HPLC, resins in H+ form, Area %), and water content, about 6% w/w, were high enough to make MVL*H₂O crystallization possible.

The crystals were filtered and centrifuged (2 min with 570 g at about 15° C.) in a filtration tube to remove mother liquid from the crystal surfaces. Thereafter these crystals and the mother liquid were analyzed. The water content of the crystals, 10.8% (by Karl-Fisher method), was a bit lower than theoretical 12.1% due to the crystallization conditions. The water content of the mother liquid was 3.2% which means that mother liquid has been concentrating during the monohydrate crystal formation. The MVL purity of the crystals was 98.8% of DS and mother liquid 97.7%/DS. These MVL*H₂O crystals were used as seed crystals in Example 14. Before being used, the crystals were grinded in a porcelain mill.

### Example 14

### Water Solvent Crystallization of Mevalonolactone Monohydrate

This example describes the water solvent crystallization of mevalonolactone monohydrate from a mevalonolactone syrup produced by subjecting sodium mevalonate crystallization mother liquor to cation exchange.

The centrifugation mother liquor from Example 6, comprising sodium mevalonate, was treated with cation exchange in accordance with Example 12 to obtain a liquid comprising mevalonolactone. The mevalonolactone purity of the resulting syrup was 71% on DS and the color was 34000 ICUMSA.

The feed syrup was evaporated to a DS of 93.7% (Rotavapor R-153 evaporator). 2.1 kg of the resulting syrup was moved to a 2-liter cooling crystallizer and seeded with 0.4 g of mevalonolactone monohydrate dry seed (prepared in accordance with Example 13) at a temperature of 11° C. The seeded syrup was cooled to a temperature of 7° C. within 16 hr under continuous stirring. After cooling to 7° C., the stirring was continued at 7° C. for 50 hr. Deionized water was added during the constant temperature period to dilute the crystal mass in the following three portions: (1) 40 g was added 23 hr after seeding, (2) 50 g was added 43 hr after seeding, and (3) 10 g was added 47 hr after seeding. The DS of the resulting crystal mass was 89.1%.

1.7 kg of the crystal mass was centrifuged in a batch-wise centrifuge without wash water (basket diameter 22.5 cm, 4000 rpm, 3 min). The mevalonolactone yield in centrifugation was 52%. The mevalonolactone purity of the centrifugation cake was 94% on DS, the water content of the non-dried cake, was 10.8 wt. %, and the color was 5200 ICUMSA. The mevalonolactone purity of the centrifugation mother liquor was 56% on DS and the color was 52000 ICUMSA.

Mevalonolactone monohydrate crystals prepared in accordance with this process were used as seed crystals in Example 15. Before being used, the crystals were grinded in a porcelain mill.

### Example 15

### Water Solvent Crystallization of Mevalonolactone Monohydrate

This example describes the water solvent crystallization of mevalonolactone monohydrate from a syrup comprising mevalonolactone produced by using nanofiltration followed by a cation exchange step.

Nanofiltration permeate prepared in accordance with Example 4, comprising sodium mevalonate, was treated with cation exchange in accordance with Example 10 to obtain a liquid comprising mevalonolactone. The mevalonolactone purity of the resulting syrup was 76% on DS and the color was 2400 ICUMSA.

The feed syrup was evaporated to a DS of 90.7% (Rotavapor R-153 evaporator). 6.3 kg of the resulting syrup was moved to a 6-liter cooling crystallizer and seeded with 0.5 g of mevalonolactone monohydrate dry seed (prepared in accordance with Example 14) at a temperature of 12 C. The seeded syrup was cooled to a temperature of 6° C. within 17 hr under continuous stirring. After cooling to 6° C., the stirring was continued at 6° C. for 5 hr. Deionized water was added during the constant temperature period at 6° C. to dilute the crystal mass in the following three portions: (1) 50 g was added 18 hr after seeding, (2) 40 g was added 19 hr after seeding. and (3) 70 g was added 20 hr after seeding. The DS of the resulting crystal mass was 88.1%.

5.2 kg of the resulting crystal mass was centrifuged in 4 batches using an amount of wash water equal to 27-33 mL/kg mass DS (batch-wise centrifuge, basket diameter 22.5 cm, 3500 rpm, 3 min). A crystal cake sample and a mother liquor sample were collected from the first centrifugation. The mevalonolactone yield in the first centrifugation was 71%. The mevalonolactone purity of the first centrifugation cake was 93% on DS, the water content of the non-dried cake was 12.7 wt. %, and the color was 560 ICUMSA. The mevalonolactone purity of the first centrifugation mother liquor was 55% on DS and the color was 5700 ICUMSA.

Mevalonolactone monohydrate crystals prepared in accordance with this process were used as seed crystals in Examples 16 and 17. Before being used, the crystals were grinded in a porcelain mill.

### Example 16

### Water Solvent Crystallization of Mevalonolactone Monohydrate

This example describes the water solvent crystallization of mevalonolactone monohydrate from a syrup comprising mevalonolactone produced by using nanofiltration followed by a cation exchange step.

Nanofiltration permeate prepared in accordance with Example 3, comprising Na-MVA, was treated with cation exchange in accordance with Example 11 to produce a liquid comprising MVL. The mevalonolactone purity of the resulting syrup was 81% on DS and the color was 1200 ICUMSA.

The feed syrup was evaporated to a DS of 84.1% (Rotavapor R-153 evaporator). 6.2 kg of the resulting syrup was moved to a 6-liter cooling crystallizer and seeded with 1.0 g of mevalonolactone monohydrate dry seed (prepared in accordance with Example 15) at a temperature of 16° C. The seeded syrup was kept at 16° C. for 3 hr and then cooled to a temperature of 6° C. within 11 hr under continuous stirring. After cooling to 6° C., the stirring was continued at 6 for 7 hr.

5.5 kg of the resulting crystal mass was centrifuged in 5 batches using an amount of wash water equal to 72-79 mL/kg mass DS (batch-wise centrifuge, basket diameter 22.5 cm, 3500 rpm, 3 min). A crystal cake sample and a mother liquor sample were collected from the first centrifugation. The mevalonolactone yield in the first centrifugation was 62%. The mevalonolactone purity of the first centrifugation cake was ≥97% on DS. the water content of the non-dried cake was 11.6 wt. %, and the color was 61 ICUMSA. The mevalonolactone purity of the first centrifugation mother liquor was 64% on DS and the color was 2700 ICUMSA.

### Example 17

### Water Solvent Crystallization of Mevalonolactone Monohydrate

This example describes the water solvent crystallization of mevalonolactone monohydrate from a mixture of centrifugation mother liquor and diluted crystal mass.

The crystallization feed material was an aqueous syrup comprising mevalonolactone. it was obtained by combining the centrifugation mother liquor and the diluted crystal mass recovered from washing the equipment with deionized water from Example 16. The mevalonolactone purity of the resulting syrup was 70% on DS and the color was 2400 ICUMSA.

The feed was evaporated to a DS of 85.3% (Rotavapor R-153), and 2.8 kg of the resulting syrup was moved to a 2-liter cooling crystallizer. The syrup was seeded two times: at first, with 1.0 g of mevalonolactone monohydrate dry seed (prepared in accordance with Example 15) at a temperature of 13° C., and then with 1.0 g of mevalonolactone monohydrate dry seed at a temperature of 10° C. The first seeding resulted in very little crystal formation.

The seeded syrup was kept at 10° C. for 35 min and then cooled to a temperature of 4 within 16 hr under continuous stirring. After cooling to 4° C., the stirring was continued at 4° C. for 2 hr.

2.5 kg of the resulting crystal mass was centrifuged in 2 batches (hatch-wise centrifuge, basket diameter 22.5 cm, 3500 rpm, 3 min). The amount of wash water in the first centrifugation was equal to 90 mL/kg DS. The resulting mevalonolactone yield in centrifugation was 42%. The mevalonolactone purity of the centrifugation cake was ≥97% on DS, the water content of the non-dried cake was 14.7 wt. %, and the color was 33 ICUMSA. The mevalonolactone purity of the centrifugation mother liquor was 58% on DS and the color was 4200 ICUMSA.

The second centrifugation was carried out without wash water. The resulting mevalonolactone yield in centrifugation was 54%. The mevalonolactone purity of the centrifugation cake was ≥97% on DS. the water content of the non-dried cake was 11.7 wt. %, and the color was 120 ICUMSA. The mevalonolactone purity of the centrifugation mother liquor was 53% on DS and the color was 4600 ICUMSA.

Mevalonolactone monohydrate crystals prepared in accordance with this process were used as seed crystals in Examples 18 and 19. Before being used. the crystals were grinded in a porcelain mill.

### Example 18

### Water Solvent Crystallization of Mevalonolactone Monohydrate

This example describes the water solvent recrystallization of mevalonolactone monohydrate crystals dissolved in deionized water.

The crystallization feed material was an aqueous syrup comprising mevalonolactone. It was obtained by dissolving and diluting 2.2 kg of centrifugation cakes from Example 15 by adding deionized water. The mevalonolactone purity of the resulting syrup was 92% on DS and the color was 560 ICUMSA.

The feed syrup was evaporated to a DS of 80.8% (Rotavapor R-153 evaporator), and 2.3 kg of the resulting syrup was moved to a 2-liter cooling crystallizer. The syrup was cooled under continuous stirring without seeding in the following two steps: (1) from 20° C. to 15° C. within 2 hr and (2) from 15° C. to 10° C. within 10 hr. After cooling to 10° C., the stirring was continued at 10° C. for 6 hr. This did not result in any spontaneous crystal formation.

The syrup was seeded with 0.5 g of mevalonolactone monohydrate dry seed (prepared in accordance with Example 17) at a temperature of 10 C. The cooling water temperature in the crystallizer jacket was kept constant at 7° C. Crystal formation started immediately after seeding. The temperature of the crystal mass increased first from 10° C. to 16° C. within 50 min from seeding due to heat of crystallization and then decreased from 16° C. to 12° C. within 3 hr. After cooling to 12° C., the cooling water temperature was increased from 7° C. to 12° C., and the stirring was continued at 12° C. for 1 hr.

2.2 kg of the resulting crystal mass was centrifuged in 2 batches using an amount of wash water equal to 65-66 mL/kg mass DS (batch-wise centrifuge, basket diameter 22.5 cm, 3500 rpm, 3 min). A crystal cake sample and a mother liquor sample were collected from the first centrifugation. The mevalonolactone yield in the first centrifugation was 52%. The mevalonolactone purity of the first centrifugation cake was ≥98% on DS. the water content of the non-dried cake was 13.6 wt. %, and the color was 35 ICUMSA. The mevalonolactone purity of the first centrifugation mother liquor was 87% on DS and the color was 1100 ICUMSA.

### Example 19

### Water Solvent Crystallization of Mevalonolactone Monohydrate

This example describes the water solvent recrystallization of mevalonolactone monohydrate crystals dissolved in deionized water.

The crystallization feed material was an aqueous syrup comprising mevalonolactone. It was obtained by combining 2.0 kg of centrifugation cakes from Example 16 with 320 g of centrifugation cakes from Example 17. Before mixing, the mevalonolactone product from. Example 16 was diluted with deionized water to a DS of 80.9%, and the resulting syrup was treated with active carbon in accordance with the procedure described in Example 6 (20 g of Norit DX 1 carbon powder per 1 kg of DS, contact time 1 hr, contact temperature 50° C., carbon powder separated by using Buchner filtration with Kenite 300 filter aid). The mevalonolactone purity of the crystallization feed syrup was ≥97% on DS and the color was 34 ICUMSA.

The feed syrup was evaporated to a DS of 78.3% (Rotavapor R-151 evaporator). 2.6 kg of the resulting syrup was moved to a 5-liter cooling crystallizer and seeded with 0.4 g of mevalonolactone monohydrate dry seed (prepared in accordance with Example 17) at a temperature of 17° C. The seeded syrup was kept at 17° C. for 1.5 hr and then cooled to a temperature of 13° C. within 15 hr under continuous stirring. After cooling to 13° C., the stirring was continued at 13° C. for 5 hr.

2.4 kg of the resulting crystal mass was centrifuged in 2 batches using an amount of wash water equal to 65-71 mL/kg mass DS (batch-wise centrifuge, basket diameter 22.5 cm, 3500 rpm, 3 min). A crystal cake sample and a mother liquor sample were collected from the first centrifugation. The mevalonolactone yield in the first centrifugation was 50%. The mevalonolactone purity of the first centrifugation cake was ≥98% on DS, the water content of the non-dried cake was 12.1 wt. %, and the color was 3 ICUMSA. The mevalonolactone purity of the first centrifugation mother liquor was 96% on DS and the color was 70 ICUMSA.

Mevalonolactone monohydrate crystals prepared in accordance with this process were used as seed crystals in Example 20. Before being used, the crystals were grinded in a porcelain mill.

### Example 20

### Water Solvent Crystallization of Mevalonolactone Monohydrate

This example describes the water solvent crystallization of mevalonolactone monohydrate and the preparation of an aqueous, high-purity, mevalonolactone syrup.

The crystallization feed material was an aqueous syrup comprising mevalonolactone. it was obtained by combining the second centrifugation cake from Example 17 with the centrifugation mother liquors from Examples 18 and 19. The syrup was diluted to a DS of a 51.2% by adding deionized water, and the resulting solution was treated with active carbon in accordance with the procedure described in Example 6 (20 g of Norit DX 1 carbon powder per 1 kg of DS, contact time 1 hr, contact temperature 50° C., carbon powder separated by using Buchner filtration with Kenite 300 filter aid). The mevalonolactone purity of the resulting syrup was 93% on DS and the color was 170 ICUMSA.

The carbon treated feed syrup was evaporated to a DS of 80.3% (Rotavapor R-153 evaporator). 2.6 kg of the resulting syrup was moved to a 2-liter cooling crystallizer and seeded with 0.6 g of mevalonolactone monohydrate dry seed (prepared in accordance with Example 19) at a temperature of 18 C. The seeded syrup was kept at 18° C. for 1 hr and then cooled to a temperature of 13° C. within 15 hr under continuous stirring. After cooling to 13° C., the stirring was continued at 13° C. for 4 hr.

2.3 kg of the crystal mass was centrifuged in 2 batches using an amount of wash water equal to 67-68 mL/kg mass DS (batch-wise centrifuge, basket diameter 22.5 cm, 3500 rpm, 3 min). A crystal cake sample and a mother liquor sample were collected from the first centrifugation. The mevalonolactone yield in the first centrifugation was 51%. The mevalonolactone purity of the first centrifugation cake was ≥98% on DS. the water content of the non-dried cake was 12.3 wt %, and the color was 3 ICUMSA. The mevalonolactone purity of the first centrifugation mother liquor was 87% on DS and the color was 370 ICUMSA.

DSC analysis of mevalonolactone monohydrate crystals prepared in accordance with this process resulted in an endothermic peak with a peak maximum at 22.6° C.

The centrifugation cakes were combined and diluted to a DS of 57.4% by adding deionized water to get liquid mevalonolactone product. The resulting syrup was evaporated to a DS of ≥97% (Rotavapor R-153 evaporator). The mevalonolactone purity of the evaporated syrup was ≥98% on OS, and the color was 2 ICUMSA.

Typical conditions for crystallizing MVL*H₂O are shown in Tables 13A-13E.

TABLE 13 A
Conditions for crystallizing MVL * H₂O with purity of feed
between 50-100% of DS and between 55-99% of DS

| | Min-Max | Preferably |
|---|---|---|
| Feed purity (% DS) | 50-100 | 55-99 |
| Temperature (° C.) | 0-24 | 0-24 |
| DS content (%) | 65-95 | 70-88 |

TABLE 13B
Conditions for crystallizing MVL* H₂O.
Purity of feed between 55-70% of DS.

| | Min-Max | Preferably |
|---|---|---|
| Temperature (° C.) | 0-20 | 0-14 |
| DS content (%) | 82-95 | 84-88 |

TABLE 13C
Conditions for crystallizing MVL* H₂O.
Purity of feed between 70-80% of DS.

| | Min-Max | Preferably |
|---|---|---|
| Temperature (° C.) | 0-22 | 0-16 |
| DS content (%) | 79-94 | 81-88 |

TABLE 13D
Conditions for crystallizing MVL* H₂O.
Purity of feed between 80-93% of DS.

| | Min-Max | Preferably |
|---|---|---|
| Temperature (° C.) | 0-24 | 3-20 |
| DS content (%) | 72-90 | 74-86 |

TABLE 13E
Conditions for crystallizing MVL* H₂O.
Purity of feed between 93-98% of DS.

| | Min-Max | Preferably |
|---|---|---|
| Temperature (° C.) | 0-24 | 3-20 |
| DS content (%) | 65-90 | 70-86 |

In addition, MVL*H₂O can be crystallized at temperatures below 0° C. down to the freezing point of the mother liquid if cooling equipment is capable and viscosity of the crystal mass is acceptable.

### Example 21

### Characterization of Mevalonolactone Monohydrate (MVL*H₂O)

Crystalline mevalonoladone monohydrate (MVL*H₂O) having the formula C₆H₁₀O₃*H₂O. was further characterized as described below.

### A. X-ray-Crystal Structure

X-ray diffraction of MVL*H₂O was measured.

### B. Melting Point

The melting point (m.p.) of MVL-monohydrate (MVL*H₂O) was between 20-25° C., preferably 21-24° C. depending on the crystal purity. Crystallization is possible only below the m.p. The melting point is determined by using Differential Scanning Calorimeter (DSC) peak temperature. DSC thermogram was measured by using Mettler Toledo DSC822e differential scanning calorimeter. The measurement was run in standard 40 μL aluminum crucible in flowing nitrogen atmosphere with a flow rate of 80 mL/min. The temperature range was 0-50° C. and the heating rate was 2° C./min.

### C. Crystalline Water Content

Theoretical water content of MVL-monohydrate is 12.15% (DS content 87.85%) when calculated from the molecular weights. (The molecular weight of MVL*H₂O is 148.16 g/mol). This corresponds with the crystals which are crystallized at the conditions having excess of water. if there is less water in the crystallization syrup than the crystal water content, the mother liquid is concentrating upon crystallization and crystalline water content of forming crystals become lower than theoretical. A crystalline water content of forming MVL-monohydrate crystals of 10.8% (by Karl-Fisher method) was observed as described in Example 13. A crystalline water content of forming MVL-monohydrate crystals of 10-11% was also observed for syrups having a high syrup DS (96 and 89).

### D. Solubility in Water

Solubility is one property to characterize a crystalline compound. Crystallization is possible only above solubility concentration in supersaturated syrups. Solubility determines minimal DS concentration for the crystallization at given temperatures. The solubility is determined by standard methods like analyzing the equilibrium concentration from crystal suspension.

The MVL*H₂O solubility in water was determined by using crystals having a purity of 93%) and calculated to purity 100. Equilibrium DS contents were measured and calculated between 0-20° C. range (Table 14).

TABLE 14
MVL* H₂O solubility

| Temperature | solubility |
|---|---|
| (° C.) | (% DS) |
| 0 | 43.9 |
| 4.8 | 52.3 |
| 9.8 | 61.2 |
| 15.0 | 70.6 |
| 20.0 | 79.7 |

### Example 22

### Effect of Mevalonolactone on Exfoliation of Skin

Exfoliation, known also as chemical peeling belong to a group of cutaneous resurfacing procedures that are used in the treatment of photoageing, inflammatory dermatoses, epidermal proliferations, pigmentary disorders and scarring (O'Conner et al. 2018, Australian Journal of Dermatology 59:171-181). Chemical peels consist of the application of one or more chemical ablative agents to the skin's surface to induce keratolysis or keratocoagulation. This process causes the controlled destruction of all or part of the epidermis or dermis. resulting in the subsequent exfoliation of these layers. This leads to the improved appearance and texture of the treated skin. pKa is an important concept in chemical peeling. It is the pH at which half of the solution is in a free acid form. A low pKa value indicates a greater availability of free acid and hence a stronger peel. Alpha hydroxy acids (AHA) peels may cause stinging, skin redness, mild skin irritation, and dryness (O′Conner et al. 2018, Australian Journal of Dermatology 59:171-181). At lower concentrations. AHA causes decreased corneocyte adhesion (Matarasso S L, Glogau R G, Markey A C. Wood's lamp for superficial chemical peels. J. Am. Acad. Dermatol. 1994; 30: 988-92.) At higher concentrations it promotes epidermolysis. (Matarasso S L, Glogau R G, Markey A C. Wood's lamp for superficial chemical peels. J. Am. Acad. Dermatol. 1994; 30: 988-92.) AHA requires neutralisation to terminate its action which can be achieved with water, sodium bicarbonate, sodium hydroxide or ammonium salt solutions.

This example describes the effect of mevalonolactone on the exfoliation (peeling) of skin compared to the effect of control compounds.

A study, such as but not limiting to, a clinical study is conducted to evaluate the chemical exfoliating effect and efficacy in improving epidermal turnover, also called cell renewal or skin regeneration, of mevalonolactone in comparison with at least one control compounds (reference compound). Control compounds include, but are not limited to, alpha hydroxy acids (AHA) such as malic acid, tartaric acid, citric acid, glycolic acid, lactobionic acid, lactic acid, malic acid, beta hydroxy acids such as salicylic acid, and retinoid family compounds such as retinol, or any combination thereof.

The active ingredients tested (mevalonolactone or control compound) are formulated in identical standard cosmetic bases, at controlled percentages (say between 0.0001% to 80% of total composition), resulting in cosmetic compositions (such as but not limited to cosmetic cream). A placebo composition, which is the cosmetic base which does not contain any active, is included as well and will serve as reference for the other actives. The base cream formula can be any formula well known to one skilled in the art.

The study is performed in 2 phases of respectively about 3 weeks and 2 weeks. The objective of Phase 1 is to assess and compare the efficacy of cosmetic compositions (comprising mevalonolactone or a control composition) on the skin redness, moisturizing and skin barrier function after repeated applications for 3 weeks, based on clinical evaluation by a trained assessor, tewarnetric and corneometric measurements.

The objective of Phase 2 is to assess and compare the efficacy on the cell renewal (exfoliation) of cosmetic compositions (comprising mevalonolactone or a control composition), after repeated applications for about 2 weeks (preceded by 3 weeks of application), on a colored skin induced by an acid such as but not limiting to DiHydroxyAcetone (DHA) based on colorimetric measurements.

The cosmetic formulations comprising the active ingredients tested (mevalonolactone or control compound) are applied to a subject by topical application. Four areas are delimited on subjects' forearms (2 treated areas, 1 untreated area acting as control area, and 1 unused area. The surface of each area is about 4×5 cm (20 cm2).

For about 5 weeks, each subject applies 2 products according to randomization, twice a day (in the morning and in the evening). under normal conditions of use (on clean skin, by slight massage until complete penetration), on delimited zones on their forearms.

Evaluation of efficacy of treatment is conducted by clinical scoring by a trained assessor, of criteria linked to the skin redness. moisturizing, skin barrier function, cell renewal (exfoliation), and any one combination thereof, before and after repeated applications of the cosmetic compositions and in comparison, with a control area (non-treated) and with values obtained before any cosmetic composition application.

### Example 23

### Effect of Mevalonolactone on Skin Barrier Function

This example describes the effect of mevalonolactone on the skin barrier function and skin barrier strengthening, compared to the effect of control compounds.

In one aspect the example includes, but is not limited to, evaluating the effect of the efficacy of mevalonolactone to moisturize the upper layers of the epidermis and/or the efficacy of mevalonolactone on the cutaneous barrier function after repeated applications.

Skin barrier strengthening or increase in skin barrier or enhancement or improvement of skin barrier function means that the skin (such as, but not limiting to, its permeability barrier) is somehow “lighter/stronger” and it limits or avoids compounds from getting in (such as microbes, pollutants etc.) but also that it limits or avoids the amount of water that gets out, i.e. the skin barrier function gets improved. One way to check if the skin barrier has been strengthened is to determine the amount of water that comes out of the skin (e.g., if skin barrier is damaged, more water is coming out).

A study, such as but not limiting to, a clinical study is conducted to evaluate and compare the skin moisturization and skin barrier function, of mevalonolactone in comparison with at least one control compounds (reference compound). Control compounds include, but are not limited to, ceramides, cholesterol, fatty acids, lanosterol, squalene, alpha hydroxy acids (AHA), beta hydroxy acids, retinoid family compounds such as retinol, or any combination thereof. Ceramides along with cholesterol and fatty acids are lipidic components of the stratum corneum and can strengthen the skin barrier as well as providing a sensorial benefit (Rogers et al. 1996 Arch Dermatol Res. 288(12):765-770, and as such can be used as positive controls in this study. Compounds that can hamper skin barrier function can be used as a negative control.

The active ingredients tested (mevalonolactone or control compound) are formulated in identical standard cosmetic bases, at controlled percentages (say between 0.0001% to 80% of total composition), resulting in cosmetic compositions (such as but not limited to cosmetic cream). A placebo composition, which is the cosmetic base which does not contain any active, is included as well and will serve as reference for the other actives. The base cream formula can be any formula well known to one skilled in the art.

The cosmetic formulations comprising the active ingredients tested (mevalonolactone or control compound) are applied to a subject by topical application. Four areas are delimited on subjects' forearms (2 treated areas, 1 untreated area acting as control area, and 1 unused area. The surface of each area is about 4×5 cm (20 cm2).

For about 2-3 weeks, each subject applies 2 products according to randomization, twice a day (in the morning and in the evening), under normal conditions of use (on clean skin, by slight massage until complete penetration), on delimited zones on their forearms.

Evaluation of efficacy of treatment is conducted by measuring the water loss of the skin and/or the moisturizing effect of the cosmetic compositions on the skin. Water loss and moisturization of the skin can be measured by any method known to one skilled in the art.

In one aspect, the water loss of the skin is determined by measurement of Trans Epidermal Water Loss (T.E.W.L. g/m²/h) which is a non-invasive method to assess the integrity of the stratum corneum barrier function, by quantifying the water exchanges between the skin and the environment (Loden M. et al., 1992, British J of Dermatol 1992; 126: 137-141). Damage of the stratum corneum results in an increase in the T.E.W.L., leading to a more pervious skin. This method enables to assess and compare the protective effect of cosmetic investigational compositions on the cutaneous barrier between themselves, after repeated applications, and in comparison, with a control area (non-treated) and with values obtained before any products application.

In one aspect, the moisturizing effect of the cosmetic compositions is determined by measuring the epidermal upper layers water content by measurements of the skin electrical capacitance and quantifying the skin moisturization. (Korstanje C. et al., 1992, J. of Dermatol. Treatment 1992; 2: 137-139; Vilaplana Jet al., 1992, Acta Derm. Venereol. 1992; 72: 28-33).

### Example 24

### Effect of Mevalonolactone on Skin Barrier Function and Skin Moisturization

This example describes the effect of mevalonolactone (MVL) on the skin barrier function and skin moisturization.

In one aspect the example includes, but is not limited to, evaluating the effect of the efficacy of mevalonolactone to moisturize the upper layers of the epidermis and/or the efficacy of mevalonolactone on the cutaneous barrier function after repeated applications.

Skin barrier strengthening means that the skin is somehow “tighter/stronger” and it limits or avoids compounds from getting in (such as microbes, pollutants etc.) but also that it limits or avoids the amount of water that gets out. One way to check if the skin barrier has been strengthened is to determine the amount of water that comes out of the skin (e.g., if skin barrier is damaged more water is coming out).

Studies were conducted to evaluate and compare the skin moisturization and skin barrier function, of mevalonoladone in comparison with a placebo treatment.

Mevalonolactone was obtained and purified as described herein and formulated in a standard cosmetic base, at 1% of total composition by weight, resulting in cosmetic a composition (such as but not limited to a cosmetic cream). A placebo cosmetic composition, which is the cosmetic base which does not contain any active (e.g. does not contain any MVL), was included in the studies as well and served as reference for the MVL active. The base cream formula can be any formula well known to one skilled in the art.

Two separated studies were performed. Study 1 consisted of measuring the skin transepidermal water loss after topical application of the cosmetic creams (a cream with 1% MVL of total composition by weight called “MVL cream” and the control cream which was the same cream without MVL called “placebo”) formulated at pH=5.5, the skin physiological pH.

Study 2 consisted of measuring the skin electrical capacitance, related to skin water content in the upper layer of the skin, after topical application of the cosmetic creams (“MVL cream” and “placebo”) formulated at pH=3.5. This pH can be described as an acidic pH and is the pH at which exfoliating products are used to induce an accelerated skin desquamation, resulting in a faster epidermal cell renewal.

### Effect of MVL on Skin Transepidermal Water Loss (TEWL) (Skin Barrier Function)

The cosmetic formulations comprising MVL as the active ingredient and the placebo treatment were applied to 6 female Caucasian subjects with dry skin (electrical capacitance<50 A.U.) by topical application. Three areas were delimited on subjects' forearms (1 treated area with “MVL cream”, 1 treated area with the “placebo”, 1 untreated area acting as control area. The surface of each area is about 4×5 cm (20 cm2). The creams applied had a pH=5.5.

For 2 weeks, each subject applied the MVL containing formulation and placebo formulation according to randomization. twice a day (in the morning and in the evening), under normal conditions of use (on clean skin, by slight massage until complete penetration), on delimited zones on their forearms.

Evaluation of the efficacy of the treatment was conducted by measuring the evolution of the transepidermal water loss of the skin induced by the cosmetic compositions on the skin. Water loss of the skin was determined by measurement of Trans Epidermal Water Loss (T.E.W.L. g/m²/h) which is a non-invasive method to assess the integrity of the stratum corneum barrier function, by quantifying the water exchanges between the skin and the environment (Loden M. et al., 1992, British J of Dermatol 1992; 126: 137-141). Damage of the stratum corneum results in an increase in the T.E.W.L., leading to a more pervious skin. This method enables to assess and compare the protective effect of cosmetic investigational compositions on the cutaneous barrier between themselves, after repeated applications. and in comparison, with a control area (non-treated) and with values obtained before any products application.

In this study, the TEWL (g/m²/h) was assessed at 5 different time points (Day 0 (baseline before product application). Day 1, Day 3, Day 7, Day 14; also named D0, D1, D3, D7 and D14).

TABLE 15
Transepidermal water toss
evolution over 2 weeks on 3 zones

| | | Δ TEWL | Δ TEWL |
|---|---|---|---|
| | Control | Placebo | MVL |
| | area | cream | Cream |
| D0 | 0 | −1.58 | 0.48 |
| D1 | 0 | −0.73 | 1.06 |
| D3 | 0 | −024 | 1.08 |
| D7 | 0 | −0.65 | 0.3 |
| D15 | 0 | −0.12 | 0.9 |

Δ TEWL = (TEWL value treated zone in g/m²/h)-(TEWL value of the control area in g/m²/h) absolute values

TABLE 16
Effect of MVL on transepidermal
water toss evolution over 2 weeks.

| | Δ TEWL |
|---|---|
| Day | MVL |
| D0 | 2.06 |
| D1 | 1.79 |
| D3 | 1.32 |
| D7 | 0.95 |
| D15 | 1.02 |

Results obtained with data from Table 15 normalized to control and placebo Δ TEWL _{MVL} = (Δ TEWL value zone “MVL cream”)-(Δ TEWL value zone “placebo cream”).

TABLE 17
Time effect of MVL on transepidermal
water loss evolution over 2 weeks

| | Δ TEWL |
|---|---|
| | MVL |
| Time | (Dx-D0) |
| D1-D0 | −0.27 |
| D3-D0 | −0.74 |
| D7-D0 | −1.11 |
| D15-D0 | −1.04 |

Results obtained with data from Table 16 normalized to control, placebo and initial time baseline D0. Δ TEWL = (Δ TEWL_{MVL} Dx)-(Δ TEWL_{MVL} D0) no unit

Table 17 indicates that MVL when topically applied as an active ingredient to the skin, decreases the skin trans epidermal water loss after 2 weeks of repeated application. The decrease in skin trans epidermal water loss can already be observed after 3 days. As such, the TEAL decrease indicates that topical applications of MVL can increase (strengthening) the skin barrier function as is evidence by the reduced water loss.

### Effect of MVL on Skin Electrical Capacitance (Skin Moisturization)

The cosmetic formulation comprising MVL as the active ingredient and the placebo treatment was applied to 5 to 11 female Caucasian subjects with normal non-sensitive skin by topical application. Three areas were delimited on subjects' forearms (1 treated area with “MVL cream”, 1 treated area with the “Placebo”, 1 untreated area acting as control area). The surface of each area is about 4×5 cm (20 cm2). The creams applied had a pH=3.5.

For 3 weeks, each subject applied the MVL formulation and the placebo formulation according to randomization, twice a day (in the morning and in the evening), under normal conditions of use (on clean skin, by slight assage until complete penetration), on delimited zones on their forearms.

Evaluation of the efficacy of the MVL treatment was conducted by measuring the water content of the skin, i.e., the moisturizing effect of the cosmetic compositions on the skin. Moisturization of the skin can be measured by any method known to one skilled in the art.

The moisturizing effect of the cosmetic compositions was determined by measuring the epidermal upper layers water content by measurements of the skin electrical capacitance with a Comeometer™ and quantifying the skin moisturization. (Korstanje C. et al., 1992, J. of Dermatol. Treatment 1992; 2: 137-139; Vilaplana Jet al., 1992, Acta Germ. Venereol. 1992; 72: 28-33).

In this study, the Electrical capacitance was assessed at 2 different time points: at baseline before product application and after 21 days; respectively named D0 and D21. Electrical capacitance is expressed in Arbitrary Units (A.U.).

TABLE 18
Effect of MVL on Electrical Capacitance of the skin.
Electrical capacitance of the 3 zones before and
after 3 weeks of MVL application: calculation of the
evolution over 3 weeks Δ (D21-D0) for each zone

| | Electrical capacitance (AU) | | |
|---|---|---|---|
| | Mean +/− standard error on the mean (S.E.M.) | | |
| | Control area | Placebo cream | MVL cream |
| | (n = 11) | (n = 5) | (n = 6) |
| D0 (initial time) | 33.75 +/− 0.68 | 31.48 +/− 1.69 | 33.32 +/− 2.45 |
| D21 (after 3 | 35.12 +/− 1.42 | 33.60 +/− 2.91 | 38.33 +/− 2.94 |
| weeks) | | | |
| Δ (D21-D0) | 1.36 +/− 1.78 | 2.12 +/− 3.08 | 5.02 +/− 1.92 |
| Probability p: | 0.462 | 0.529 | 0.048 |
| Time effect | | | |
| Student “t” test | | | |
| % increase of | | | 15%^{#} |
| AU of MVL | | | |
| treated skin | | | |
| after 3 weeks | | | |

^{#}Statistically significant probability: <0.05
^{#}variation with regards to D0

Table 18 clearly indicates that topical application of the MVL containing cosmetic cream significantly increased skin electrical capacitance over time, as evidenced by a 15% increase after 3 weeks of repeated application. As such, this data indicates that MVL helps to increase the water content of the skin and moisturizes the skin by improving the water retention, thereby providing an increased skin moisturization. In comparison, the time effect of the placebo cream was not significant versus initial time. The time effect of MVL cream on skin electrical capacitance was significantly higher than that of the placebo cream, proving that the moisturizing effect over time is due to MVL as an ingredient as such.

TABLE 19
Variation of electrical capacitance over time
Δ (D21-D0), normalized to control area

| Variations of | | | |
|---|---|---|---|
| electrical capacitance | Control | Placebo | MVL |
| (Arbitrary units) | area | cream | cream |
| D0 (initial time) | 0 | −2.27 | −0.43 |
| D21 (after 3 weeks) | 0 | −1.52 | 3.21 |
| Δ (D21-D0) creams | 0 | 0.75 | 3.64 |

The data of Table 19 was obtained as follows: Normalization to control of the electrical capacitance (A.U.) of the placebo zone at baseline=D0 placebo−D0 control area
Normalization to control of the electrical capacitance (A.U.) of the MVL cream zone at baseline=D0 MVL cream−D0 control area.
Normalization to control of the electrical capacitance (A.U.) of the placebo zone after 3 weeks=D21 placebo−D21 control area.
Normalization to control of the electrical capacitance (A.U.) of the MVL cream zone after 3 weeks=D21 MVL cream−D21 control area.

Normalization to control of the variation of the electrical capacitance over time: A (D21−D0) creams=[Δ(D21−D0) creams]−[A(D21−D0) control area]=Variation of the electrical capacitance of the MVL cream or placebo cream zone over time−Variation of the electrical capacitance of the control area over time.

Normalization is aimed at removing the “noise” results measured on the control area to calculate the “treatment” results starting from the same baseline and allows fair comparison of the products effects (MVL effect) among the group of volunteers. By doing so one does remove from the measured water content, the amount of water that was already present in the skin naturally without any product application. Hence, we can measure the amount of water that is brought, retained or generated following the cream application (placebo or MVL). As the amount of water content in the skin can differ from one panelist to the other, a normalization is required to remove the variation due to the panelists skin's uniqueness.
A(D21−D0) creams: Variation of electrical capacitance of the creams zones over 3 weeks
Effect of the placebo cream after 3 weeks: Δ (D21−D0) placebo=D21 placebo−D0 placebo
Effect of the MVL cream after 3 weeks: Δ (D21−D0) MVL cream=D21 MVL cream−D0 MVL cream

Table 19 indicates that the moisturizing effect of MVL cream was higher than the placebo cream. As such, MVL increased the skin water content, which is consistent with the observed increase of the skin barrier function (TEFL decrease) described in Table 17.

### Example 25

### Effect of Mevalonolactone on Skin Barrier Function

A test referred to as “caffeine diffusion test” was performed, which consists of the measurement of the effect of compounds mevalonolactone (MVL) on the skin barrier reinforcement. It is one method to measure skin barrier function (strength) as when the skin barrier has been strengthened, the amount of penetrating tracer molecules decreases (i.e. if skin is damaged more, the easier the tracer penetrates the skin). In this study, the effect of mevalonolactone, was assessed on [¹⁴C]-caffeine diffusion through reconstructed human epidermis (RHE) under basal condition, in order to evaluate the skin barrier function. Caffeine diffusion is inversely proportional to barrier function. RHE (RHE, 0-day-old, Bioalternatives reference: EPI-Ba, batch n° 01015-515) were cultured under the following conditions: 37° C., 5% CO2, and the culture media was the Bioaltematives maintenance medium. The tested MVL concentration were 0.001%, 0.005% and 0.01% by weight of assay medium, in systemic way (added to the culture media).

### Culture and Treatment

RHE at day 0 were placed at the air/liquid interface in culture medium containing or not (control) the test compounds, their association or the reference (vitamin C at 200 μg/ml) (systemic application). RHE were then, incubated for 7 days with treatment renewals at day 3 and day 5. All experimental conditions were performed in n=3.

### Caffeine Diffusion

At the end of incubation (D7), radioactive caffeine ([¹⁴C]-caffeine) was applied onto the surface of each epidermis. The radioactivity contained in subnatants was measured by liquid scintillation according to the following kinetic: 30 minutes, 1 and 2 hours.

### Data Management

Raw data were analyzed using Microsoft Excel® software and GraphPad PRISM® software. The inter-group comparisons were performed by an unpaired Student's t-test. The statistical analysis can be interpreted if n≥5, however for n<5 the statistical values are for information only. Formulas used in this report:

Standard error of the mean: sem=Sd/√n; Barrier function:

$$Barrier {function}_{( \% Control )} = \frac{Mean [ \frac{1000}{{cpm}_{sample}} ]}{Mean [ \frac{1000}{{cpm}_{Control}} ]} \times 100$$

### Results

Caffeine diffusion through control reconstructed human epidermis (RHE) under non-treated condition was progressive during the whole kinetic. The systemic treatment of RHE with vitamin C, tested at 200 μg/ml, decreased significantly this diffusion, thus highlighting an increase of epidermal barrier function. These results were expected and validated the assay.

TABLE 20
Effect of the compounds Mevalonolactone on barrier function of RHE.
Measurement of the [¹⁴C]-caffeine diffusion through RHE after 30 minutes,
1 and 2 hours of incubation.

| | | 30 min | | | 1 hour | | | 2 hours | | |
|---|---|---|---|---|---|---|---|---|---|---|
| Treatment | | | | Barrier | | | Barrier | | | Barrier |
| Test | | Mean | % | function | Mean | % | function | Mean | % | function |
| compound | Conc. | (cpm) | control | (% control) | (cpm) | control | (% control) | (cpm) | control | (% control) |
| Control | — | 19360 | 100 | 100 | 51299 | 100 | 100 | 115547 | 100 | 100 |
| Vitamin C | 200 | 11133 | 58 | 171** | 34839 | 68 | 148** | 90717 | 79 | 128** |
| | microg/ | | | | | | | | | |
| | ml | | | | | | | | | |
| MVL | 0.001% | 14907 | 77 | 128 | 48423 | 94 | 106 | 117165 | 101 | 99 |
| | 0.005% | 16927 | 87 | 113 | 55034 | 107 | 93 | 122324 | 106 | 95 |
| | 0.01% | 19700 | 102 | 97 | 53545 | 104 | 96 | 116509 | 101 | 99 |

*0.01 to 0.05, Significant, **0.001 to 0.01

As shown in Table 20, the systemic treatment of the RHE with mevalonolactone, tested at 0.001%, showed a trend for a decreased diffusion of caffeine through the RHE (MVL 0.001%: starting at 128% of control after 30 mins, 106% of control after 1 hour and 99% of control) suggesting that MVL had an effect on the skin barrier improvement.

This can also be explained by an improved skin desquamation effect, indeed, the desquamation process is a bottom to top process, whereas caffeine diffusion is going from top to bottom (caffeine being deposited on the skin model surface). Desquamation improvement will tend to slow down the active (caffeine) penetration by promoting the cell turnover and renewal fro e epidermal basal layer to the upper epidermal layers.

### Example 26

### Effect of Mevalonolactone on Lipidic Composition of Skin

This example describes the effect of topically applied mevalonolactone on the skin lipid composition of a skin model tissue compared to the effect of untreated skin model.

The effects of mevalonolactone (MVL) on lipidomic profile of the skin was assessed by using a commercial Phenion® Full-Thickness (FT) Skin Model (Henkel, Henkel AG & Company, Germany). The FT skin model was treated in vitro with topical MVL and compared with FT skin model treated with a control solution without MVL, and after the treatment of 24 hours, the skin tissues were lysed, and lipids were extracted from the tissues. Total lipidomics (total lipid profile) was performed utilizing LC-MS, and the lipid profile of tissues treated with MVL were compared to the lipid profile of tissues that were not treated, with MVL.

### Tissues

The full-thickness skin models contain both epidermal keratinocyte and dermal fibroblast cells. Tissues were placed on top of the Phenion® Air Liquid lnterphase Culture Medium (ALI-Medium, ALI CM-250, Henkel, Henkel AG & Company, Germany) soaked filter paper (on filter spacers in petri dish) in pre-warmed ALI-medium for subsequent culture at the air liquid interphase. The tissues were left over night at +37° C. in 5% CO₂ atmosphere. The tissues used in this model were Phenion® FT skin model tissues with a state of differentiation of Air-liquid-interphase (ALI) day 10, a production batch of Phe-HM-18-23, a matrix batch of Phe-Mx-18-11 and a cell batch of Phe-FB-12-06 (Henkel, Henkel AG & Company, Germany)

### Test Substances

MVL was produced and purified as described herein=MVL was in the form of a liquid syrup containing less than 5% water. Test solution was made by diluting MVL in DBPS. MVL was used with Phenion® skin models as 13% solutions (Phenion® tissues with 20 μl volume for tissue size of 1.56 cm²).

MVL test solution was pH-adjusted with 50% NaOH (Fluka) to match the cell/tissue culture medium pH to preserve the cell/tissue viability, and sterile filtered with 0.2 μm filter before use.

### Experiment Setup

The experiment was done with three replicates. Control samples were treated topically with Dulbecco's phosphate-buffered saline (DPBS) without magnesium and calcium and did not contain MVL.

MVL samples were prepared by diluting MVL in DPBS solution just before initiation of the experiment and topically applied in 20 μl volume on top of the Phenion® tissues and placed to the incubator for 24 hours at +37° C., in 5% CO₂ atmosphere. After the test substance exposure tissues were placed on Petri dishes and rinsed with DPBS filling and emptying the tissue 10 times to remove any residual test material. Tissues were then lysed and frozen for later protein concentration measurement and shipping for lipidornic analysis.

Control sample tissue was cut in half and each half was transferred with forceps into a Precellys bead tube (CK14&CK28 mix, cat no. 03961-1-009.2, Bertin Technologies, Montigny-le-Bretonneux, France) filled with 750 μl DPBS solution. The sample was treated with a Precellys 24 homogenizer (Bertin Technologies, Montigny-le-Bretonneux, France) at 5000 rpm for 3 rounds of 30 s and keeping the tube in ice for 2 minutes between the rounds.

The MVL treated samples were handled as follows: bead tube was filled with 700 μl of DPBS, the tissue was divided into two and the halves were transferred into separate tubes. Tissues were homogenized with the Precellys 24 homogenizer 3 times for 30 s at 5000 rpm (samples were kept on ice for 2 min between the rounds). The tissue lysates from divided samples were thereafter combined to one tube. Total lysis volume for control sample was 1.5 ml and for the rest of the samples 1.4 ml.

Small subsamples from the homogenized cell lysates were separated for protein concentration measurement and stored for a short period at −20° C. One ml aliquot of the tissue lysates was stored for a short period at −80° C. before sending for lipidomic analysis to Lipotype GmbH.

Cell lysate protein concentrations were determined with a Pierce BCA Protein Assay Kit (23225, Thermo Fisher Scientific) according to manufacturer's instructions. Each sample was analyzed in triplicates. Protein concentration of the unknown samples were determined with UV spectrometry and using bovine serum albumin as the calibration standard.

Mass spectrometry-based lipid analysis was performed by Lipotype GmbH (Dresden, Germany) as described in (Sampaio J L et al., Proc Natl Acad Sci USA 2011, 108:1903-7). Lipids were extracted using a two-step chloroform/methanol procedure (Ejsing C S, et al., Proc Natl Acad Sci USA 2009; 106: 2136-41). Samples were spiked with internal lipid standard mixture containing: cardiolipin 16:1/15:0/15:0/15:0 (CL), ceramide 18:1;2/17:0 (Cer), diacylglycerol 17:0/17:0 (DAG), hexosylceramide 18:1;2/12:0 (HexCer), lyso-phosphatidate 17:0 (LPA), lyso-phosphatidylcholine 12:0 (LPG), lyso-phosphatidylethanolamine 17:1 (LPE), lyso-phosphatidylglycerol 17:1 (LPG), lyso-phosphatidylinositol 17:1 (LPI), lyso-phosphatidylserine 17:1 (LPS), phosphatidate 17:0/17:0 (PA), phosphatidylcholine 17:0/17:0 (PC), phosphatidylethanolamine 17:0/17:0 (PE), phosphatidylglycerol 17:0/17:0 (PG). phosphatidylinositol 16:0/16:0 (P1), phosphatidylserine 17:0/17:0 (PS), cholesterol ester 20:0 (CE), sphingomyelin 18:1;2/12:0;0 (SM), triacylglycerol 17:0/17:0/17:0 (TAG) and cholesterol D6 (Chol). After extraction, the organic phase was transferred to an infusion plate and dried in a speed vacuum concentrator. 1st step dry extract was re-suspended in 7.5 mM ammonium acetate in chloroform/methanol/propanol (1:2:4, V:V:V) and 2nd step dry extract in 33% ethanol solution of methylamine in chloroformimethanol (0.003:5:1; V:V:V). All liquid handling steps were performed using Hamilton Robotics STARlet robotic platform with the Anti Droplet Control feature for organic solvents pipetting. Samples were analyzed by direct infusion on a QExactive mass spectrometer (Thermo Scientific) equipped with a TriVersa NanoMate ion source (Advion Biosciences). Samples were analyzed in both positive and negative ion modes with a resolution of Rm/z=200=280000 for MS and Rm/z=200=17500 for MSMS experiments, in a single acquisition. MSMS was triggered by an inclusion list encompassing corresponding MS mass ranges scanned in 1 Da increments (Surma M A, et al., European journal of lipid science and technology: EJLST 2015; 117:1540-1549). Both MS and MSMS data were combined to monitor CE, DAG and TAG ions as ammonium adducts; PC, PC O—, as acetate adducts; and CL, PA, PE, PE O—, PG, PI and PS as deprotonated anions. MS only was used to monitor LPA, LPE, LPE O—, LPI and LPS as deprotonated anions; Cer, HexCer, SM, LPC and LPC O— as acetate adducts and cholesterol as ammonium adduct of an acetylated derivative (Liebisch G, et al., Biochim Biophys Acta 2006; 1761:121-128) Data were analyzed with Lipotype GmbH in-house developed lipid identification software based on LipidXplorer (Herzog R, et al., Genome Biology 2011; 12:R8; Herzog R, et al. LipidXplorer: a software for consensual cross-platform lipidomics. PLoS One 2012; 7:e29851). Data post-processing and normalization were performed using Lipotype GmbH in-house developed data management system. Only lipid identifications with a signal-to-noise ratio>5, and a signal intensity 5-fold higher than in corresponding blank samples were considered for further data analysis. Data were analyzed with R version 3.5.1 (2018-07-02) (Team RC. R: A Language and Environment for Statistical Computing. Vienna, Austria: R Foundation for Statistical Computing, 2017.) using tidyverse packages (version 1.2.1) (Wickham H. Tidyverse: Easily Install and Load the ‘Tidyverse’, 2017) (and bioconductor pcaMethods (Stacklies W, et al., Bioinformatics 2007; 23:1164-1167). For pairwise comparisons, a non-parametric Wilcoxon tests (Mann-Whitney test) was applied to every lipid feature. P-values were adjusted for multiple testing with the Benjamini & Hochberg (1995) method are treated as significant, if p(Benjamini & Hochberg method)<0.05.

The effect of the MVL treatment on specific skin lipid classes is shown in Table 21. An increase in the amount of a lipid class by MVL is indicated by a “+”, a decrease in the amount of a lipid class by MVL is indicated by a “−” and if no difference between the MVL treated skin and the control was observed, it was indicated by a “=”. A “*” indicates a statistically significant increase or decrease at significant probability: p<0.05.

TABLE 21
Effect of MVL treatment on skin lipid groups and classes.

| | | MVL |
|---|---|---|
| | LIPID CLASS | treatment |
| LIPID Groups | Abreviation-name | effect |
| ST-Sterols | Chol-Cholesterol | = |
| | Ratio Chol/ | = |
| | Cholesterol sulfate | |
| | CE-Cholesterol Esters | + |
| SP-Sphingolipids | CER-Ceramide | = |
| | HexCer-Hexosylceramide* | + |
| | SM-Sphingomyelin | = |
| Acyl glycerols | DAG-Diacylglycerol | − |
| | TAG-Triacylglycerol | + |
| | TAG 47:2;0 | |
| PG/CL | Phosphatidylglycerol | − |
| | PG 18:1;0_18:2;0*; | |
| | 16:1;0_18:1;0*; | |
| | 16:1;0_20:2;0*; | |
| | 16:0;0_18:1;0*; | |
| | 18:2;0_20:2;0*; | |
| | 16:1;0_18:2;0* | |
| | LPG-lyso-PG | − |
| | CL-CardioLipin* | − |
| PA-Phosphatidate | | + |
| (PA and LPA | | |
| PCs-Phosphatidylcholines | LPC | = |
| | LysoPhosphatidylcholine | |
| | PC Phosphatidylcholine | + |
| | PC 15:1;0_16:0;0* | |
| | PC-O- Phosphatidylcholine- | + |
| | ether | |
| | PC-O 16:1;0/18:2;0 | |
| | (plasmalogen) | |
| PEs- | LPE* | − |
| Phosphatidylethanolamines | LPE-0 | − |
| | PE 18:1;0_18:1;0 * | − |
| | 14:0,0_18:1,0,* | |
| | 18:1;0_19:2;0;* | |
| | 18:1;0_20:2;0;* | |
| | 17:1;0_22:3;0;* | |
| | 16:1;0_18:1;0;* | |
| | 16:0;0_18:1;0;* | |
| | 16:0;0_18:2;0* | |
| | PE O-16:1;0/18:1;0* | − |
| PS-Phosphatidylserine | LPS-lyso-Phosphatidylserine | = |
| | PS-Phosphatidylserine | = |
| PI-Phosphatidylinositol | LPI-lyso phosphatidylinositol | = |
| | PI-Phosphatidylinositol | − |

The initial lipid class composition of the skin tissue samples revealed that he major lipid dasses in the tissue samples were triacylglycerol (TAG), phosphatidylcholine (PC) and cholesterol (Chol).

As shown in Table 21, MVL treated skin tissue showed a significant increase in hexosylceramide (HexCer), cholesterol esters (CE), triacylglycerol (TAG), phosphatidylcholines (PC), phosphatidate (PA), phosphatidylcholine-ether (PC-O), versus control samples: while MVL treated skin tissue showed a significant decrease in diacylglycerol (DAG), PG/CL, phosphatidylethanolamine-ethers (LPE, LPE-O, PE-O) and phosphatidylinositol (P1). Cholesterol, ratio cholesterolichol sulfate, ceramides, sphingomyelin (SM), phosphatidylserine (PS) levels were unchanged.

The topical application of MVL induced an increase in TAG (Table 21). Epidermal triacylglycerol (TAG) plays an important role in metabolism during formation of a functional permeability barrier in the skin barrier function (Radner F. P. W. and J. Fisher, Biochim Biophys Acta, 2014 March: 1841(3):409-15). Taken together, the increase in TAG observed with a topical treatment of MVL on skin (Table 21) further provides a positive impact on the skin barrier function. When the skin barrier function is improved, the skin is better protected from external aggressions like the penetration of harmful substance (xenobiotics; i.e. pollutants particles), microbes, germs and viruses. The skin is also better protected against water loss, leading to an increased water content of the skin and thus an increased skin moisturization

As shown in Table 21, the topical application of MVL induced reduction in lipids necessary for mitochondrial inner membrane (cardiolipin, CL) but did not increase, nor decrease the cholesterol biosynthesis, indicating that glucose may be preferably transformed into DHAP, rather than acetylCoA (precursor of HMG-CoA, mevalonate and cholesterol) and moves down in the lipid metabolism pathway (Lipid Metabolism in Mammals (ISBN 978-1-4684-2832-2), Author: Snyder Fred, 1977, pp 1-33, Introduction: General Pathways in the Metabolism of Lipids in Mammalian Tissues, Golde, L. M. G et al) leading to an increase of PA, TAG and PC, and decrease of DAG (precursor of TAG) and PE (precursor of PC). This suggest that there is a preferred impact of MVL on the energy production by the mitochondria. In the cytosol, HMG-CoA is converted into mevalonate, while in mitochondria, it is converted into acetyl CoA and acetoacetate. There is a competition between these 2 pathways and topical application of MVL seems to push the metabolism toward one preferred pathway: the one happening in mitochondria. This can lead to the production of CoQ10 (ubiquinone) instead of cholesterol. CoQ10 is a strong anti-oxidant which protect cell structures against the ROS generated during the mitochondria respiration and energy production process by the cell. Mitochondria plays a significant role in skin aging and CoQ10 is a personal care ingredient well known for its skin energizing and anti-aging properties.

In summary, as described above the topical application of MVL on full-thickness skin model induces a change in the skin lipid profile. Without being bound to theory, it is believed that this study of the lipid metabolism pathway in human skin shows that these changes indicate that the lipid metabolic pathway is directed towards a preferred direction which seems to be linked to the mitochondria metabolism and ubiquinone production. This further suggests a positive impact on the mitochondria energy/anti-oxidant production balance which may have a positive impact as well on the skin cells (keratinocytes and fibroblasts) metabolism global improvement. In the skin, keratinocytes are the main present in the epidermis. Keratinocytes, like all other types of cells are consuming oxygen, water and amino-acids (among other simple elements) to produce proteins, energy and CO2. This energy production is taking place within the keratinocyte's mitochondria. If mitochondria metabolism is improved. keratinocytes will benefit from a higher energy and protein production level, they are likely to produce more keratin, and to differentiate faster into corneocytes. This is called the cell turnover. The differentiation process of keratinocytes into corneocytes includes a step of lipid production and exogenesis to form the intercorneocyte lipid layer which make the skin to be a waterproof barrier. If the keratinocytes have an improved metabolism (as described above), they will also produce more Natural Moisturizing Factor from filaggrin, which participates in water retention within the skin. When the skin is well-moisturized, the lipid barrier well-formed and cell-turnover optimized, this has positive consequences on the skin mechanical and biological properties leading to an improved healthy look, also called anti-aging effect.

By reaching the dermis and stimulating dermal cells metabolism MVL can also stimulate the dermis matrix protein and glycosaminoglycans production by fibroblasts. This having a positive effect on skin dermis and dermal epidermal junction density and support for a higher mechanical strength and resistance to the effect of gravity with age. Avoiding skin to relax with aging and by consequence providing benefits like, improved skin elasticity, skin firmness, a better defined face oval, decrease sagging, decrease the appearance wrinkles for an overall improved healthier and younger skin look.

Taken together with our results described in Example 27, these results indicate that topical applications of MVL and/or MVA can result in lipid changes in the skin which can result into skin benefits like: skin moisturizing (maintaining or increasing the water content of the epidermis, and, protecting the skin against dehydration by maintaining, restoring and/or strengthening the skin barrier function), skin regeneration, skin renewal, improving epidermal cell turnover, preventing or retarding the appearance of the signs of aging of the skin, reducing the appearance of skin wrinkles, skin rejuvenation, strengthening the skin barrier function, and any one combination thereof.

### Example 27

### Effect of Mevalonic Acid & Mevalonolactone on Lipidic Composition of Skin and Skin Exfoliation (Desquamation)

Topical application of mevalonic acid (MVA) in the exact same test condition as above described for Example 26, showed a decrease of the cholesterolicholesterol sulfate ratio, which is directly influencing the keratinocytes desquamation (exfoliation) process (Table 22).

TABLE 22
Effect of MVA treatment on skin Hold qroups and classes.

| | | MVA |
|---|---|---|
| LIPID Groups | LIPID CLASS | treatment effect |
| ST-Sterols | Ratio Cholesterol/ | − |
| | Cholesterol sulfate | (p = 0.088) |

Topically applied MVL is metabolized in the skin into Na-MVA, which is in balance with MVA in aqueous solution. As such, topically applied MVL can have the same effects as topically applied MVA. but with a different kinetic subject to the molecule bioavailability (time of the molecule to be processed within the skin structures). Both MVA and MVL were introducing similar effects in lipid species or lipid classes in normal adult skin Phenion™ model. MVA treatment was found to decrease the cholesterol to cholesterol sulphate ratio, (Table 22). As the cholesterol amount was found to stay stable with the treatment, this indicates that the amount of cholesterol sulfate increased in the MVA treated skin.

Cholesterol sulfate is present in small quantities in the stratum corneum, declining from 5% of the total lipid content in the underlying layers of the SC to only 1% in the most superficial layers.(Starr et al., Analytical chemistry, 2016; Elias and al., Mol. Cell Biol. Lipids 2014, 1841, 353-361). Despite this, it has emerged as a significantly important component, with Sato et al. proposing that it plays a role in regulating desquamation, through inhibition of proteases which facilitate corneocyte loss (Sato, Denda, J. Invest. Dermatol. 1998, 111, 189-193). Williams and Elias had previously demonstrated a 5-fold increase in this molecule in patients with recessive X-linked ichthyosis (Williams, Elias et al., J. Clin. Invest. 1981, 68, 1404-10). This hereditary condition results from a deficiency in steroid sulfatase and causes extreme scaling of the skin and both Elias et al. (Elias et al. J. Clin. Invest. 1984, 74, 1414-21) and Sato et al. (Sato, Denda et al., J. Invest. Dermatol. 1998, 111, 189-193) have induced scaling of murine skin through topical cholesterol sulfate application. It is also known that this sterol is found in keratinizing membranes at much higher levels than in mucosal membranes, further supporting the suggestion that it is involved the regulation of desquamation (Elias and al., Mol. Cell Biol. Lipids 2014, 1841, 353-361). An increase in cholesterol sulfate has been associated with diseases that cause heightened desquamation, producing symptoms of scaly and peeling skin. These symptoms are also commonly seen with both elderly skin and skin which has suffered prolonged UV exposure.

Increasing the amount of cholesterol sulfates by topical application of MVA, and indirectly MVL, may have positive effect on the regulation of the skin desquamation process. Indeed, decreases in cholesterol sulphate have been noted with age (De Paepe K, Weerheim A, Houben E, et al. Skin Pharmacology & Physiology 2004; 17:23-30.). At the same time, cholesterol sulfate induces the expression of the skin barrier protein filaggrin in normal human epidermal keratinocytes. Epidermal differentiation is a key process in the skin. Basal cells are differentiating while moving up to the skin surface, into different skin layers: stratum spinosum, stratum granulosum, stratum lucidum, stratum corneum. This is called the cell turnover. Improving cell differentiation and regulating cell desquamation leads to a proper skin function and appearance (i.e. healthy look). Skin functions are multiples: mechanical protection, immune protection, protection against environment aggressors, UV protection, sensory protection (heat, cold via the sensors). Filaggrin is at the same time a marker of keratinocytes differentiation, and a precursor protein for the elements of the N.M.F. (Natural Moisturizing Factor) which retains water in the upper layers of the skin. With age, there is a decrease in filaggrin expression. With this, MVL is showing an anti-aging effect.

### Effect of MVL on Skin Desquamation (Cell Renewal)

A cosmetic formulation comprising 1% MVL (in weight) as the active ingredient and the placebo treatment was applied to 5 to 11 female Caucasian subjects with normal non-sensitive skin by topical application, aged 30-60 years old. Three areas were delimited on subjects' forearms (1 treated area with “MVL cream”. 1 treated area with the “Placebo”, 1 untreated area acting as control area). The surface of each area is about 4×5 cm (20 cm²). The creams applied had a pH=3.5.

For 5 weeks, each subject applied the MVL formulation and the placebo formulation according to randomization, twice a day (in the morning and in the evening), under normal conditions of use (on clean skin, by slight massage until complete penetration), on delimited zones on their forearms.

After an initial phase of 3 weeks of repeated application of the test products (creams), the skin of the panelist was colored by the application of Dihydroxyacetone (semi-occlusive patch) which is tanning the skin in red-orange, followed by 2 extra weeks of repeated application of the same test products (creams). Application and removal of the DHA under patches was performed as followed. On the 2 out of 3 areas of 20 cm² (5×4 cm), located to the left and right forearms (inner side) previously delimited, coloring of each area with a preparation containing 150 μl of DHA (10% solution), applied under semi-occlusive patch (2 successive times during about 3 hours and every 3 hours).

Evaluation of the efficacy of the MVL treatment was conducted by measuring the color of the skin before the DHA patch application (D0T0), and after the application of the patch, immediately (D0T3h, D0T9h) and at defined time points during a 2-weeks period after the patch application (D2, D4, D8, D9, D11, D14). Color of the skin can be measured by any method known to one skilled in the art. The cell renewal effect of the cosmetic compositions was determined by measuring the skin color on the basis of colorimetric measurements with a Chromameter™ CR400 (opening 8 mm) in the center of the 2 out of 3 areas previously delimited on the forearms. 2 measurements were performed per area. The parameters analyzed were the Light variable L*, chromaticity coordinate a* (on a green-red axis), Individual Typological Angle I.T.A.° (calculated parameter). I.T.A. is calculated as follow: ITA=[Arc tan((L*−50)/b*)]×180

The evaluation of skin cell renewal, on the basis of chromametric measurements made after staining of the skin by the method described by Pierard and Piérard-Franchimont (Piérard G. E, Piérard-Franchimont C., Dihydroxyacetone Test as a Substitute for the Dansyl Chloride Test, Dermatology 1993; 186: 133-137), allowed to objectively assess and compare the efficacy of cosmetic products, after repeated applications, between themselves and in comparison with a control area (non-treated). The decrease in intensity of staining of the skin, induced by the application of DHA, may indeed be correlated to the rate of cell renewal of the surface layers of the epidermis, compared to a colored area but without treatment by any product.

Colorimetric measurements (Chromameter™), after repeated applications of cosmetic investigational products, give colorimetric information, correlated with the cutaneous pigmentation state, and thus enable to objectively assess and compare the effect on these parameters between themselves and in comparison, with a control area (non-treated).

A statistical analysis was performed from the data, obtained. Determination of the mean values of the different criteria/parameters, at each time point of the study, on the concerned areas, by the calculation of the means and the standard errors on the mean (S.E.M.) for instrumental evaluation and standard deviations (Sd) for clinical evaluation, of individual data. Time effect: For each area, comparison of the data obtained at the considered time points to initial values, on each area.

### Product Effect:

Comparison of the data obtained, at the considered time points to initial values. on each area (control and treated areas); of all the areas between themselves (control and treated areas), at each time point of the study; of all the areas between themselves (control and treated areas) on the differences Δ(Tx−T0). In case of statistically significant evolution, calculation of the corresponding variation percentage, from the mean values, with a significance threshold of 5%.

During the repeated application of the test product on the DHA-colored area, the color of the skin is fading away. This test aims at comparing the speed at which the skin color is recovering to a light colored-skin identical to the initial skin color before the patch application. When the color measured is significantly different than the initial skin color, that means the cell renewal is not complete. When the difference is not significant anymore, that means the skin color is back to what is was before the patch application and by consequence that the cell renewal is complete.

TABLE 23
Light variable L* values (Means and
standard errors on the mean (S.E.M.)

| | | Light variable L* | | |
|---|---|---|---|---|
| | | Means and standard errors on the mean (S.E.M.) | | |
| | | Control | Cream | Cream |
| | | area | MVL | Placebo |
| | | n = 11 | n = 6 | n = 5 |
| D0T0 | | 64.96 ± 0.81 | 66.23 ± 0.97 | 64.96 ± 1.22 |
| D2 | | 54.59 ± 1.16 | 56.46 ± 0.60 | 54.02 ± 1.99 |
| D4 | | 55.29 ± 1.08 | 57.85 ± 1.20 | 55.74 ± 1.83 |
| D7 | | 57.76 ± 1.26 | 59.21 ± 1.25 | 58.00 ± 1.93 |
| D9 | | 60.22 ± 1.02 | 61.02 ± 1.08 | 61.20 ± 1.17 |
| D11 | | 61.60 ± 0.77 | 63.61 ± 0.75 | 62.38 ± 1.02 |
| D14 | | 62.89 ± 0.93 | 65.04 ± 0.77 | 63.06 ± 0.79 |
| Probability | D2 | <0.001 | <0.001 | 0.003 |
| p: Time | | #: −16% | #: −15% | #: −17% |
| effect | D4 | <0.001 | 0.002 | 0.005 |
| Student | | #: −15% | #: −13% | #: −14% |
| “t” test | D7 | <0.001 | 0.002 | 0.009 |
| | | #: −11% | #: −11% | #: −11% |
| | D9 | <0.001 | 0.003 | 0.041 |
| | | #: −7% | #: −8% | #: −6% |
| | D11 | <0.001 | 0.010 | 0.049 |
| | | #: −5% | #: −4% | #: −4% |
| | D14 | 0.001 | 0.024 | 0.017 |
| | | #: −3% | #: −2% | #: −3% |

# variation with regard to D0T0

TABLE 24
Chromaticity coordinate a* values
(Means and standard errors on the mean (S.E.M.)

| | | CHROMATICITY COORDONATE A* | | |
|---|---|---|---|---|
| | | Means and standard errors on the mean (S.E.M.) | | |
| | | Control | Cream | Cream |
| | | area | MVL | Placebo |
| | | n = 11 | n = 6 | n = 5 |
| D0T0 | | 7.84 ± 0.48 | 7.02 ± 0.57 | 7.80 ± 0.75 |
| D2 | | 13.09 ± 0.48 | 12.25 ± 0.55 | 13.78 ± 0.63 |
| D4 | | 12.55 ± 0.50 | 11.09 ± 0.63 | 12.69 ± 0.43 |
| D7 | | 11.08 ± 0.51 | 10.41 ± 0.65 | 11.35 ± 0.73 |
| D9 | | 10.17 ± 0.50 | 9.91 ± 0.61 | 9.69 ± 0.34 |
| D11 | | 9.37 ± 0.44 | 8.49 ± 0.40 | 9.80 ± 0.66 |
| D14 | | 8.83 ± 0.42 | 7.96 ± 0.35 | 9.52 ± 0.80 |
| | D2 | <0.001 | 0.003 | 0.003 |
| | | #: +67% | #: +75% | #: +77% |
| Probability | D4 | <0.001 | 0.008 | 0.005 |
| p: Time | | #: +60% | #: +58% | #: +63% |
| effect | D7 | <0.001 | 0.012 | 0.008 |
| Student | | #: +41% | #: +48% | #: +46% |
| “t” test | D9 | <0.001 | 0.011 | 0.099 |
| | | #: +30% | #: +41% | #: +24% |
| | D11 | <0.001 | 0.069 | 0.013 |
| | | #: +20% | #: +21% | #: +26% |
| | D14 | 0.004 | 0.142 | 0.013 |
| | | #: +13% | | #: +22% |

# variation with regard to D0T0

TABLE 25
Individual Typological Angle I.T.A.°
(Means and standard errors on the mean (S.E.M.))

| | | Individual Typological Angle I.T.A.° | | |
|---|---|---|---|---|
| | | Means and standard errors on the mean (S.E.M.) | | |
| | | Control | Cream M2 | Cream P2 |
| | | area | (ref:M2) | (ref: P2) |
| | | n = 11 | n = 6 | n = 5 |
| D0T0 | | 46.11 ± 2.62 | 48.20 ± 2.65 | 47.19 ± 3.64 |
| D2 | | 11.27 ± 2.90 | 15.74 ± 2.40 | 10.22 ± 5.04 |
| D4 | | 12.86 ± 2.77 | 20.42 ± 4.79 | 14.85 ± 4.93 |
| D7 | | 19.87 ± 3.43 | 25.11 ± 5.03 | 22.17 ± 5.34 |
| D9 | | 27.91 ± 3.20 | 30.96 ± 4.51 | 33.44 ± 3.89 |
| D11 | | 33.40 ± 2.36 | 38.90 ± 2.94 | 37.66 ± 3.00 |
| D14 | | 38.22 ± 2.50 | 44.64 ± 2.17 | 41.43 ± 2.15 |
| Probability | D2 | <0.001 | <0.001 | 0.003 |
| p: Time | | #: −76% | #: −67% | #: −78% |
| effect | D4 | <0.001 | 0.001 | 0.004 |
| Student | | #: −72% | #: −58% | #: −69% |
| “t” test | D7 | <0.001 | 0.003 | 0.005 |
| | | #: −57% | #: −48% | #: −53% |
| | D9 | <0.001 | 0.006 | 0.041 |
| | | #: −39% | #: −36% | #: −29% |
| | D11 | <0.001 | 0.019 | 0.046 |
| | | #: −28% | #: −19% | #: −20% |
| | D14 | 0.001 | 0.080 | 0.020 |
| | | #: −17% | #: −7% | #: −12% |

# variation with regard to D0T0

Comparisons were made as to how fast the test products (MVL cream and placebo cream) allowed the natural color to fully recover (Table 27 and 28) compared to the control zone with no product applied after the DHA patch removal (Table 26).

Data from Table 23, 24 and 25 are calculated from the values measured and reported in tables 26, 27 and 28. Variations percentages named ΔD2, ΔD4, ΔD7, ΔD11 and ΔD14 in columns of tables 26-28 are respectively calculated from data of Tables 23-25 as follows: ΔD2=D2−D0 T0; ΔD4=D4−D0T0; ΔD7=D7−D0T0; ΔD11=D11−D0T0; ΔD14=D14−D0T0.

DHA effect at D2 (Table 26, 27 and 28), ΔD2 DHA: Statistically significant decrease in Light Variable L* and and statistically significant increase in a*, for all measured area 2 days after patch removal of DHA patches (D2), in comparison with the initial values (before DHA application, D0T0), showing into evidence the effects of DHA on the skin coloration.

Time effect: Statistically significant variation of L*, a* and I.T.A.* for all the areas (control and treated), after 4 and 7 days of use, in comparison with the initial values (before DHA application). No statistically significant variation on some of the parameters analyzed, for the cream placebo and MVL cream, after 9, 11 and 14 days, in comparison with the initial values (before DHA). The color fading will induce, decrease in a* parameter (less red), increase in L* parameter (light) and increase in I.T.A° angle and a reduction of the difference measured compared to initial values (before DHA application)

TABLE 26
Variation percentage^{#} of colorimetric parameters of skin
control area and their significance versus initial measurement
(before DHA application at D0T0)

| | Control area (DHA alone) n = 11 | | | | | |
|---|---|---|---|---|---|---|
| | ΔD2 | ΔD4 | ΔD7 | ΔD9 | ΔD11 | ΔD14 |
| L* | −16% | −15% | −11% | −7% | −5% | −3% |
| a* | +67% | +60% | +41% | +30% | +20% | +13% |
| I.T.A.° | −76% | −72% | −57% | −39% | −28% | −17% |

All data in Table 23 were statistically significant at probability p<0.05; with regard to the values obtained at D0T0.

TABLE 27
Variation percentage^{#} of colorimetric parameters of “MVL cream”
area and their significance versus initial measurement (before DHA
application at D0T0)

| | MVL cream area n = 6 | | | | | |
|---|---|---|---|---|---|---|
| | ΔD2 | ΔD4 | ΔD7 | ΔD9 | ΔD11 | ΔD14 |
| L* | −15%* | −13%* | −11%* | −8%* | −4%* | −2%* |
| a* | +75%* | +58%* | +48%* | +41%* | +21% | NS |
| I.T.A.° | −67%* | −58%* | −48%* | −36%* | −19%* | −7% |

”*” indicates statistically significant at probability p < 0.05;
Probability close to significance: 0.05 ≤ p < 0.10:
NS: not significant p ≥ 0.10; ;
^{#}variation with regard to the values obtained at D0T0.

TABLE 28
Variation percentage^{#} of colorimetric parameters of “placebo cream”
area and their significance versus initial measurement (before DHA
application at D0T0)

| | Placebo cream area n = 5 | | | | | |
|---|---|---|---|---|---|---|
| | ΔD2 | ΔD4 | ΔD7 | ΔD9 | ΔD11 | ΔD14 |
| L* | −17%* | −14%* | −11%* | −6%* | −4%* | −3%* |
| a* | +77%* | +63%* | +46%* | +24% | +26%* | +22%* |
| I.T.A.° | −78%* | −69%* | −53%* | −29%* | −20%* | −12%* |

”*” indicates statistically significant at probability p < 0.05;
Probability close to significance: 0.05 ≤ p < 0.10:
NS: not significant p ≥ 0.10; ;
^{#}variation with regard to the values obtained at D0T0.

Initially (D0T0), the skin is white. After DHA application, the skin is colored (tanned) in red-orange. At this point (D2), there is a large color variation between the color measured at D2 (red skin color) and the color measured before DHA at D0T0 (white skin). A highly significant difference between the color value at D2 and D0T0 was observed (Table 27). After the DHA patch is removed, the color of the skin will then progressively come back to its initial white color over time, inducing a decrease in the color variation between the color measured at Dx and at D0T0. The speed of the color recovery of the skin is directly linked to the cell renewal rate. We assessed whether the product application on tanned skin was reducing the color variation faster than on the zone without any product applied (natural skin renewal process) or compared to the placebo without active. Starting D2, over time, the color variation was getting smaller up to a point where the difference with the initial color D0T0 was getting not significant anymore, meaning the skin color was totally recovered to its initial state. In this test, we determined at which time point the color difference (variation Dx-D0) was not significant anymore.

The earlier a non-significant difference is reached, the more efficient the product is on skin cell renewal.

Tables 26-28 indicate that the color difference compared to the initial time (D0T0, before DHA) generated by the repeated application of MI cream was non-significant on D11 and D14 on 2 parameters (a* and I.T.A.°), whereas for the placebo and control area, the difference was still significant at D11 and D14. The placebo was more efficient than control area and MVL starting D9 but the difference was significant again at D11 and D14. Taken together, these results showed the effect of MVL cream on color fading, cell renewal, was faster and more sustainable than the placebo cream.

Taken together with our results described in Examples 23-25, these results indicate that topical applications of MVL can result in improved cell renewal in the skin epidermis. which can result into skin benefits like: skin surface and irregularities smoothing, also called skin resurfacing (via a better regulation of skin desquamation), skin regeneration, skin renewal, improving epidermal cell turnover, preventing or retarding the appearance of the signs of aging of the skin, reducing the, appearance of skin wrinkles, skin rejuvenation, strengthening the skin barrier function, and any one combination thereof. Improved skin desquamation helps to remove the dead cells that tends to accumulate on the skin surface with age when the cell turnover decrease. Stratum corneum thickness is decreased leading to a less rough skin surface and nicer looking skin. Skin resurfacing effect, leading to a smoother skin surface participates to a better and more homogeneous light reflection and give a nice brighter and more radiant and even skin tone.

What is claimed is:

1. A skin care composition for providing at least one skin care benefit in a subject, comprising an effective amount of mevalonolactone, wherein said composition provides at least one skin care benefit to said subject.

2. The skin care composition of claim 1, further comprising one or more dermatologically or cosmetically acceptable components.

3. The skin care composition of claim 1, wherein said effective amount of mevalonolactone is between 0.01% to 10.0% on a weight basis relative to a total weight of said composition.

4. The skin care composition of claim 1, wherein the at least one skin care benefit is selected from the group consisting of skin moisturizing, skin exfoliation (also referred to as skin peeling, desquamation, skin shedding), skin resurfacing, skin regeneration, skin renewal, improving epidermal cell turnover, preventing or retarding the appearance of the signs of aging of the skin (anti-aging), reducing the appearance of skin wrinkles, skin rejuvenation, strengthening the skin barrier function, and any one combination thereof.

5. The skin care composition of claim 1, wherein the at least one skin care benefit is strengthening the skin barrier function.

6. The skin care composition of claim 1, wherein the at least one skin care benefit is skin moisturization.

7. The skin care composition of claim 1, wherein the at least one skin care benefit is skin exfoliation.

8. The skin care composition of claim 1, wherein said composition changes the lipidic profile of the skin of said subject.

9. The skin care composition of claim 1, wherein the composition is selected from the group consisting of an aqueous solution, an emulsion, a serum, a jelly, a mask, a patch, a face mask, a peel-off mask, a lotion, a topical moisturizer, a cream, a paste, a balm, an ointment, a pomade, a gel, a liquid, a spray, a foam, a kits, a sun care, a baby care, a hair care, and any one combination thereof,

10. A method for providing at least one skin care benefit in a subject, comprising contacting a skin of said subject with a skin care composition comprising an active ingredient selected from the group consisting of mevalonolactone, mevalonic acid, mevalonolactone monohydrate, and any one combination thereof.

11. The method of claim 10, wherein the at least one skin care benefit is selected from the group consisting of skin moisturizing, skin exfoliation (also referred to as skin peeling. desquamation, skin shedding), skin resurfacing, skin regeneration, skin renewal, improving epidermal cell turnover, preventing or retarding the appearance of the signs of aging of the skin (anti-aging), reducing the appearance of skin wrinkles, skin rejuvenation, strengthening the skin barrier function, and any one combination thereof.

12. A method for strengthening the skin barrier function of a skin in a subject, said method comprising contacting a skin of said subject with a skin care composition comprising an effective amount of an active ingredient selected from the group consisting of mevalonolactone, mevalonic acid, mevalonate, mevalonolactone monohydrate, or any one combination thereof, wherein said effective amount of said active ingredient results in an improved skin barrier function versus a control skin than was contacted with a placebo composition lacking said effective amount of said active ingredient.

3. A method for providing an increased skin exfoliation to a skin of a subject, said method comprising contacting a skin of said subject with a skin care comprising an effective amount of an active ingredient selected from the group consisting of mevalonolactone, mevalonic acid, mevalonate. mevalonolactone monohydrate, or any one combination thereof, wherein said effective amount of said active ingredient result in an increased skin exfoliation versus a control skin than was contacted with a placebo composition lacking said effective amount of said active ingredient.

14. A method for increasing the skin moisturization of a skin in a subject, said method comprising contacting a skin of said subject with a skin care composition comprising an effective amount of an active ingredient selected from the group consisting, of mevalonolactone, mevalonic acid, mevalonate, mevalonolactone monohydrate, or any one combination thereof, wherein the skin moisturization of the skin contacted with said composition is increased versus a control skin than was contacted with a placebo composition lacking said effective amount of said active ingredient.

15. The method of anyone of claims 10-14, wherein said skin care composition further comprises one or more dermatologically or cosmetically acceptable component.

16. The method of anyone of claims 10-14, wherein the effective amount of the active ingredient is at least 0,01%, 0.02%, 0.03%, 0.04%, 0.05%, 0.06%, 0.07%, 0.08%, 0.09%, 0.1%, 0.2%, 0.3%, 0.5%, 0.6%, 0.7%, 0.8%, 0.9%, 1%, 2%, 3%, 4%, 5%, 6%, 7%, 8%, 9% up to 10% on a weight basis relative to a total weight of said composition.
